(19) United States
(12) Patent Application Publication
(10) Pub. No.: US 2026/0090966 A1
(43) Pub. Date: Apr. 2, 2026

(54) UROLITHIN COMPOSITIONS

(71) Applicant: Amazentis SA, Lausanne (CH)

(72) Inventors: Christopher L. Rinsch, Lausanne (CH); Davide D'Amico, Renens (CH); Anurag Singh, Lausanne (CH); Charlotte Tissot, Lausanne (CH)

(21) Appl. No.: 19/288,549

(22) Filed: Aug. 1, 2025

(30) Foreign Application Priority Data
Aug. 8, 2024 (GB) 2411739.2

(51) Int. Cl. A61K 8/49; A61K 8/21; A61K 8/22; A61K 8/24; A61K 8/25; A61K 8/27; A61K 8/34; A61K 8/73; A61K 8/9789; A61K 31/366; A61P 1/02; A61P 29/00; A61Q 11/00

(52) U.S. Cl. CPC A61K 8/498; A61K 8/21; A61K 8/22; A61K 8/24; A61K 8/25; A61K 8/27; A61K 8/34; A61K 8/73; A61K 8/9789; A61K 31/366; A61P 1/02; A61P 29/00; A61Q 11/00; A61K 2800/28

(57) ABSTRACT

The invention relates to compositions comprising urolithins, specifically oral care compositions which have a therapeutic or cosmetic effect in the oral cavity, for example, the teeth, gums cheek, tongue etc. Such oral compositions include, toothpaste and gels, mouthwash, mouth rinses and mouth sprays and other oral care products, such as dental floss/tape, dental strips, dental sticks and chewing gum. The invention further comprises oral compositions comprising further active ingredients, for example, fluoride. The invention further comprises methods of using such compositions and formulations in the treatment of conditions and diseases and for cosmetic uses and processes for the preparation of such compositions. The invention also comprising methods of reducing the toxic effects of additives to oral composition, such as fluoride.

### RELATED APPLICATION

This application claims the benefit of priority to UK Patent Application No. GB 2411739.2, filed Aug. 8, 2024.

### BACKGROUND

Oral care products such as toothpastes can provide both therapeutic and cosmetic hygiene benefits. Therapeutic benefits include caries prevention which is typically provided by the use of various fluoride salts; gingivitis prevention by the use of antimicrobial agents; or hypersensitivity control. Cosmetic benefits provided by oral products include the control of plaque and calculus formation, removal and prevention of tooth stain, tooth whitening, breath freshening, and overall improvements in mouth feel impression.

One differentiating factor among oral care products, such as toothpaste, is the active ingredient, fluoride. The most commonly used fluoride sources are typically sodium fluoride (NaF), sodium monofluorophosphate, and stannous fluoride. Sodium fluoride and sodium monofluorophosphate are effective sources of fluoride ions that remineralize and strengthen weakened enamel thus allowing fighting cavities. By comparison, stannous fluoride not only delivers cavity fighting fluoride, but it also has antibacterial properties, and it provides an anti-sensitivity mechanism of action. Stannous fluoride has both bactericidal and bacteriostatic properties, which fight plaque and treat/prevent gingivitis. Stannous fluoride also deposits a protective mineral barrier over exposed dentinal tubules to help prevent sensitivity pain from triggers such as hot or cold liquids and foods. However, exposure of human gingival fibroblasts to fluoride and its salts, such as Sodium Fluoride (NaF), results in increased cellular damage and mitochondrial dysfunction in different cell types derived from the oral cavity. This raises the potential of developing interventions to prevent potentially toxicological effects of fluoride on oral health (Stepniak et al (2014) Int J Nanomedicine 9, 1677-1687; PMC3979695). For examples, treatment of human gingival cells with fluoride led to increased reactive oxygen species (ROS) production, oxidative stress, reduced cell viability, and mitochondrial dysfunctions (Stepniak et al (2014) ibid, PMC3979695). Co-exposure with fluoride and silver nanoparticles (AgNPs)—also commonly used in dental practise, led to even more pronounced cellular health dysfunctions (Stepniak et al (2014) ibid, PMC3979695).

Another study confirmed that NaF induces apoptosis in human gingival fibroblasts (HGF) through both the mitochondria-mediated pathways regulated by the Bcl-2 family and death receptor-mediated pathway (Lee et al (2008) Toxicology 243(3), 340-47, PMID 18069112).

Fluoride was shown to negatively impact cellular health also in other cell types in the oral cavity. For instance, NaF is used to promote enamel mineralization (Aulestia et al (2020) Sci Signal 13(619); PMID: 32071168). However, NaF treatment of enamel cells was shown to negatively affect mitochondrial respiration, elicit mitochondrial membrane depolarization, and disrupt mitochondrial morphology (Aulestia et al (2020) Sci Signal 13(619); PMID: 32071168).

Hydrogen peroxide (H₂O₂) is another active commonly used in oral care practises for teeth whitening. However, H₂O₂ is a well-established inducer of reactive oxygen species (ROS) and has been shown to induce a variety of cellular dysfunctions when exposed to gingival cells. For instance, H₂O₂ exposure to gingival cells significantly induced ROS already after 1 hour and with increased detrimental effects with exposure up to 24 hours (Zgorzynska et al (2015) Arch Oral Biol 60(1) 144-53; PMID: 25455128). Chronic exposure to H₂O₂ in the same cell line was also associated with increased cellular senescence and production of pro-inflammatory cytokines and reduction of collagen production (Furukawa et al (2022) Clin Exp Dent Res 8(4) 939-949; PMC9382052).

In summary, a number of active ingredients present in oral care products may lead to cellular stress, often resulting in increased cellular inflammation. In addition, oral cavity tissues undergo a natural aging process that also contributes to low-grade chronic inflammation, known as “inflamm-aging” (Franceschi et al., 2017, Trends Endocrinol Metab, 28(3):199-212; PMID: 27789101). The combination of natural aging and exposure to actives with adverse effects on cell health makes inflammation a key hallmark of oral cavity health.

A common biomarker of inflammation in this context is the pro-inflammatory cytokine interleukin-6 (IL-6), which has been associated with age-related changes such as frailty (PMID: 35054284; Gomez-Rubio et al (2022) Diagnostics (Basel) 12(1):117) and is also elevated in periodontitis (Gaetano et al (2021) Archives of oral biology122, 104997; PMID: 33291049). Large meta-analyses have identified IL-6 as the most prevalent cytokine in the saliva of children and young adults with active dental caries (Alarcón-Sánchez et al (2024) BMC Oral Health 24(1):816; PMID: 39026257). In this latter case, inflammation, measured as salivary IL-6 concentration, was associated with dental caries with changes as small as a 10% increase in IL-6 levels (Table 3, Alarcón-Sánchez et al (2024) ibid; PMID: 39026257).

To further support the clinical relevance of such cytokines, agents like thymol, a well-known antiseptic used to reduce gingivitis, have demonstrated health benefits associated with reductions in cytokine levels in gingival crevicular fluid, including interleukin-1 (IL-1), by 14% to 23% (Amoian et al (2017) Electronic Physician 9(9), 5223-5228; PMID: 10207532).

In conclusion, elevated salivary interleukins, including IL-6 and IL-1, are validated biomarkers of gingival health (Isola et al (2021) Arch Oral Biol. 122; 104997; PMID: 33291049), and active ingredients capable of reducing their secretion represent promising strategies for improving oral health.

Urolithins have been proposed as treatments for a variety of conditions related to inadequate mitochondrial activity, including obesity, reduced metabolic rate, metabolic syndrome, diabetes mellitus, cardiovascular disease, hyperlipidaemia, neurodegenerative diseases, cognitive disorders, mood disorders, stress, and anxiety disorders; for weight management, or to increase muscle performance or mental performance. See WO2012/088519 (Amazentis SA). In WO2007/127263 (The Regents of the University of California), the use of urolithins for the treatment of various neoplastic diseases is described.

International patent publication WO2014/004902 (derived from application PCT/US2013/48310) discloses a method of increasing autophagy, including specifically mitophagy, in a cell, comprising contacting a cell with an effective amount of a urolithin or a pharmaceutically acceptable salt thereof, thereby increasing autophagy, including specifically mitophagy, in the cell. Administration may be to a subject having a disease or condition selected from metabolic stress, cardiovascular disease, endothelial cell dysfunction, sarcopenia, muscle degenerative disease, Duchenne muscular dystrophy, alcoholic liver disease, non-alcoholic fatty liver disease, drug-induced liver or muscle injury, al-antitrypsin deficiency, ischemia/reperfusion injury, inflammation, aging of the skin, inflammatory bowel disease, Crohn's disease, obesity, metabolic syndrome, type II diabetes mellitus, hyperlipidaemia, osteoarthritis, neurodegenerative disease, Alzheimer's disease, Huntington's disease, Parkinson's disease, amyotrophic lateral sclerosis, age-related macular degeneration, mitochondrial diseases (including for example poor growth, loss of muscle coordination, muscle weakness, visual problems, hearing problems, heart disease, liver disease, kidney disease, gastrointestinal disorders, respiratory disorders, neurological problems, autonomic dysfunction sometimes learning disabilities, and dementia (as a result of mitochondrial disease), muscle diseases; cancer, cognitive disorder, stress, and mood disorder.

### SUMMARY

The invention relates to compositions comprising urolithins, specifically oral care compositions which have a therapeutic or cosmetic effect in the oral cavity, for example, the teeth, gums cheek, tongue etc. Such oral compositions include, toothpaste and gels, mouthwash, mouth rinses and mouth sprays and other oral care products, such as dental floss/tape, dental strips, dental sticks and chewing gum. The invention further comprises oral compositions comprising further active ingredients, for example, fluoride. The invention further comprises methods of using such compositions and formulations in the treatment of conditions and diseases and for cosmetic uses and processes for the preparation of such compositions.

Surprisingly, it has been found that, in addition to the uses described above, it is advantageous to include urolithins in oral care compositions. For example, to enhance mitophagy in the oral cavity.

According to a one aspect of the invention there is provided an oral care composition comprising:
a) a compound of formula (I) or a salt thereof:

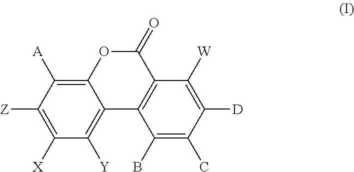

wherein:
A, B, C and D are each independently selected from H and OH;
W, X and Y are each independently selected from H and OH; and
Z is selected from H and OH.

According to one aspect of the invention there is provided an oral care composition comprising:
a) a compound of formula (I) or a salt thereof, as defined herein; and
b) an oral care composition additive, wherein the oral care composition additive is selected from one or more of a fluoride source, a fluoride replacement, an antiseptic and a whitening agent.

Examples of oral care compositions include toothpaste, powder, mouthwash, oral sprays, oral gels, mouth rinses, chewing gum, and lozenges, for example, toothpaste, oral gels or mouthwash, for example toothpaste.

### BRIEF DESCRIPTION OF THE FIGURES

FIG. 1: Cell viability of human gingival fibroblasts (HGFs) treated with sodium fluoride (NaF) for 24 hours at a 3 mM dose. Data are expressed as % change compared to DMSO and are mean+/−SEM. ****p<0.0001 t-test.

FIG. 2: Cell viability of HGF pretreated with Urolithin A at the indicated doses for 24 hours, then co-treated with both sodium fluoride (NaF) 3 mM and Urolihin A for additional 24 hours. Data are mean+/−SEM.*p<0.05, ***p<0.001, ****p<0.0001 One-way ANOVA.

FIG. 3: IL-6 secretion by HGF pretreated with Urolithin A at the indicated concentrations for 24 hours, followed by co-treatment with 3 mM sodium fluoride (NaF) and Urolithin A for additional 24 hours. IL-6 levels in supernatants were measured by ELISA. Data are expressed as pg/mL and are mean+/−SEM.**p<0.01, ***p<0.001, ****p<0.0001 One-way ANOVA.

FIG. 4: Comparison of IL6 secretion by ELISA in human gingival fibroblasts (HGF) at low (P4) and late (P8) passages. Data are expressed as pg/mL and are mean±SEM. ****p<0.0001 unpaired t-test.

FIG. 5: IL6 secretion in HGF at low (P4) and late (P8) passages after UA treatment. HGF P8 cells were treated with control (DMSO) or the indicated doses of Urolithin A for 48 hours. IL6 levels in cell supernatants were measured by ELISA. Data are expressed as pg/mL and are mean±SEM. **p<0.01 ****p<0.0001 One-way ANOVA.

FIG. 6: IL-6 secretion by HGF pretreated with Urolithin A at the indicated concentrations for 24 hours, followed by co-treatment with H₂O₂ 400 uM and Urolithin A for an additional 24 hours. IL-6 levels in cell supernatants were measured by ELISA. Data are expressed as pg/mL and are mean+/−SEM.*p<0.05, ****p<0.0001 One-way ANOVA.

FIG. 7: IL-6 secretion by HGF pretreated with Urolithin A at the indicated concentrations for 24 hours, then exposed 2 hours to H₂O₂ and, after replacement of media without H₂O₂, incubated for 5 days with either DMSO or Urolithin A at the indicated doses. IL-6 levels in cell supernatants were measured by ELISA. Data are expressed as pg/mL and are mean+/−SEM.*p<0.05, **p<0.01 One-way ANOVA.

FIG. 8: IL-6 secretion measured by ELISA in human gingival fibroblasts exposed 30 min to NaF at different doses. Then, after replacement of culture medium without NaF, cells were incubated for an additional 24 hours. Data are expressed as pg/ml. Data are mean+/−SEM.**p<0.01, ****p<0.0001 One-way ANOVA.

FIG. 9A: IL-6 secretion by human gingival fibroblasts pretreated with urolithin A at the indicated concentrations for 24 hours, followed by 30 minutes exposure to NaF 5 mM (A), and—following replacement of culture medium without NaF, subsequent treatment with urolithin A for additional 24 hours. IL-6 levels were measured by ELISA. Data are mean+/−SEM. ***p<0.001, ****p<0.0001 One-way ANOVA.

FIG. 9B: IL-6 secretion by human gingival fibroblasts pretreated with urolithin A at the indicated concentrations for 24 hours, followed by 30 minutes exposure to NaF 10 mM(B) and—following replacement of culture medium without NaF, subsequent treatment with urolithin A for additional 24 hours. IL-6 levels were measured by ELISA. Data are mean+/−SEM. ***p<0.001, ****p<0.0001 One-way ANOVA.

FIG. 9C: IL-6 secretion by human gingival fibroblasts pretreated with urolithin A at the indicated concentrations for 24 hours, followed by 30 minutes exposure to NaF 50 mM (C) and—following replacement of culture medium without NaF, subsequent treatment with urolithin A for additional 24 hours. IL-6 levels were measured by ELISA. Data are mean+/−SEM. ***p<0.001, ****p<0.0001 One-way ANOVA.

FIG. 10: Human dermal fibroblasts (HDFs) pretreated with urolithin A (UA) 2.5 uM, hydroxyapatite (HA) 500 uM, the combination of UA 2.5 uM and HA 500 uM, and DMSO as control (−) for 24 hours. After 24 h, cells were treated with DMSO (−) of co-treated with an inflammatory cytokine mix (Cytomix, CTX) alone and with CTX in combination with urolithin A and HA at the doses indicated. After additional 24 h, IL-6 levels were measured by ELISA. Data are mean+/−SEM. ***p<0.001, ****p<0.0001 One-way ANOVA.

FIG. 11: IL-6 secretion measured by ELISA in Human gingival fibroblast (HGF) treated for 48 hours with either menthol 1.5 mM, Urolithin A 10 uM or their combination. Data are mean+/−SEM. *p<0.05. One-way ANOVA.

### DETAILED DESCRIPTION

According to a one aspect of the invention there is provided an oral care composition comprising:
a) a compound of formula (I) or a salt thereof:

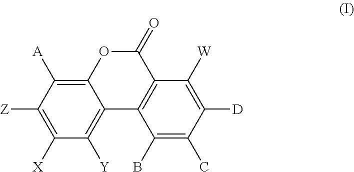

wherein:
A, B, C and D are each independently selected from H and OH;
W, X and Y are each independently selected from H and OH; and
Z is selected from H and OH.

According to one aspect of the invention there is provided an oral care composition comprising:
a) a compound of formula (I) or a salt thereof, as defined herein; and
b) an oral care composition additive, wherein the oral care composition additive is selected from one or more of a fluoride source, a fluoride replacement, an antiseptic and a whitening agent.

Examples of oral care compositions include toothpaste, powder, mouthwash, oral sprays, oral gels, mouth rinses, chewing gum, and lozenges, for example, toothpaste, oral gels or mouthwash, for example toothpaste.

According to a further aspect of the invention, there is provided an oral care composition, comprising:
a) a compound of formula (I) or a salt thereof, as defined herein; and
b) a fluoride source.

Examples of suitable fluoride sources, include fluoride salts, for example, sodium fluoride, stannous fluoride, potassium fluoride, potassium stannous fluoride, indium fluoride, zinc fluoride, ammonium fluoride, stannous chlorofluoride, sodium monofluorophosphate, sodium fluorosilicate, ammonium fluorosilicate, amine fluoride, titanium fluoride, hexafluorosulphate, or a mixture of two or more thereof. Further examples of suitable fluoride sources can be found in U.S. Pat. Nos. 3,535,421, 4,885,155, and 3,678,154.

Examples of further fluoride sources include sodium fluoride, potassium fluoride, disodium monofluorophosphate, tin(II)fluoride, dipotassium fluorophosphates, calcium fluorophosphates, calcium fluoride, ammonium fluoride, aluminium fluoride, hexadecyl ammonium fluoride, 3-(N-hexadecyl-N-2-hydroxy-ethylammonio) ammonium difluoride, N,N-N-Tris(polyoxyethylene)-N-hexadecyl-propylenediaminedihydrofluoride disodium hexafluorosilicate, dipotassiumhexafluorosilicate, ammonium hexafluorosilicate, magnesium hexafluorosilicate or ammonium fluorophosphates, or any combinations of two or more thereof.

More commonly used fluoride sources are typically sodium fluoride, sodium monofluorophosphate, and stannous fluoride. Sodium fluoride and sodium monofluorophosphate are effective sources of fluoride ions that remineralize and strengthen weakened enamel thus allowing fighting cavities. By comparison, stannous fluoride not only delivers cavity fighting fluoride, but it also has antibacterial properties, and it provides an anti-sensitivity mechanism of action. Stannous fluoride has both bactericidal and bacteriostatic properties, which fight plaque and treat/prevent gingivitis. Stannous fluoride also deposits a protective mineral barrier over exposed dentinal tubules to help prevent sensitivity pain from triggers such as hot or cold liquids and foods.

According to a further aspect of the invention, there is provided an oral care composition, essentially free of a fluoride source.

According to a further aspect of the invention, there is provided a oral care composition, comprising:
a) a compound of formula (I) or a salt thereof;
b) a fluoride source; and
c) an abrasive.

According to a further embodiment of the invention, there is provided an oral care composition of the invention, wherein the composition is applied to an oral care product.

According to a further embodiment of the invention, there is provided an oral care composition of the invention, wherein the composition is incorporated into the matrix of an oral care product.

Examples of oral care products include dental floss, dental tape, dental strips and dental sticks (tooth picks).

According to a further aspect of the invention, there is provided an oral care composition, comprising:
a) a compound of formula (I) or a salt thereof, as defined herein; and
b) a fluorine replacement.

Examples of fluoride replacements include arginine, xylitol, citric acid, zinc citrate, activated charcoal, calcium sodium phosphosilicate (Novamin), casein phosphopeptide-amorphous calcium phosphate (CPP-ACP), and hydroxyapatite, for example, calcium hydroxyapatite.

According to a further aspect of the invention, there is provided an oral care composition, comprising:
a) a compound of formula (I) or a salt thereof, as defined herein; and
b) an antiseptic.

Examples of antiseptics include thymol, eucalyptol, menthol, and/or methyl salicylate.

In a further embodiment, an antiseptic is selected from one or more of essential oils (e.g., rosemary extract, tea extract, magnolia extract, thymol, menthol, eucalyptol, geraniol, carvacrol, citral, hinokitol, miswak extract, sea-buckthorn extract), and/or one or more zinc salts.

In a further embodiment the antiseptic is menthol.

According to a further aspect of the invention, there is provided an oral care composition, comprising:
a) a compound of formula (I) or a salt thereof, as defined herein; and
b) a whitening agent.

Examples of whitening agents include a peroxide source, such as hydrogen peroxide, sodium peroxide and/or carbamide peroxide.

### Formulations

According to a further aspect of the invention, there is provided an oral care composition, for example, a toothpaste, comprising:
a) about 0.1% to about 10% (w/w) of a compound of formula (I) as defined herein, for example, about 0.5% ot about 2% (w/w) or about 1% (w/w) for example, urolithin A; and
b) about 25 to about 25,000 ppm of a fluoride source, for example, about 500 to about 5000 ppm or about 1500 ppm, for example, sodium fluoride; and
c) one or more inactive carriers.

According to a further aspect of the invention, there is provided an oral care composition, for example, a toothpaste, comprising:
a) about 0.1% to about 10% (w/w) of a compound of formula (I) as defined herein,
b) for example, about 0.5% ot about 2% (w/w) or about 1% (w/w) for example, urolithin A; about 25 to about 25,000 ppm fluoride, for example, about 500 to about 5000 ppm or about 1500 ppm, for example, sodium fluoride; and
c) one or more of the following components:
   i) about 0.005% to about 1% (w/w) PQQ, for example, about 0.01% to about 0.1% (w/w) or about 0.05% w/w;
   ii) about 0.05% (w/w) to about 5% (w/w) ginseng extract, or one or more ginsenosides, for example, about 0.5% to about 2% (w/w) or about 1% (w/w);
   iii) about 0.05% (w/w) to about 5% (w/w) notoginseng extract, or one or more notoginsenosides, for example, about 0.5% to about 2% (w/w) or about 1% (w/w);
   iv) about 0.01% to about 5% (w/w) zinc ion source, for example, about 0.1% to about 1% (w/w) or about 0.2% (w/w), for example, zinc citrate and/or zinc oxide;
   v) about 0.01% to about 5% (w/w) hyaluronic acid, for example, about 0.1% to about 1% (w/w) or about 0.3% (w/w);
   vi) about 0.01% to about 5% (w/w) pectin, for example, about 0.1% to about 1% (w/w) or about 0.3% (w/w);
   vii) about 0.05% to about 5% (w/w) quercetin, for example, about 0.5% to about 2% (w/w) or about 1% (w/w); and
d) one or more inactive carriers.

According to a further aspect of the invention, there is provided an oral care composition, for example, a toothpaste, comprising:
a) about 0.1% to about 10% (w/w) of a compound of formula (I) as defined herein, for example, about 0.5% ot about 2% (w/w) or about 1% (w/w) for example, urolithin A; and
b) about 1% to about 50% (w/w) of a fluoride replacement, for example, about 5% to about 20% (w/w) or about 10% (w/w); and
c) one or more inactive carriers.

According to a further aspect of the invention, there is provided an oral care composition, for example, a toothpaste, comprising:
a) about 0.1% to about 10% (w/w) of a compound of formula (I) as defined herein, for example, about 0.5% ot about 2% (w/w) or about 1% (w/w) for example, urolithin A; and
b) about 1% to about 50% (w/w) hydroxyapatite, for example, about 5% to about 20% (w/w) or about 10% (w/w); and
c) one or more inactive carriers.

According to a further aspect of the invention, there is provided an oral care composition, for example, a toothpaste, comprising:
a) about 0.1% to about 10% (w/w) of a compound of formula (I) as defined herein, for example, about 0.5% ot about 2% (w/w) or about 1% (w/w) for example, urolithin A;
b) about 1% to about 50% (w/w) hydroxyapatite, for example, about 5% to about 20% (w/w) or about 10% (w/w); and
c) one or more of the following components:
   i) about 0.005% to about 1% (w/w) PQQ, for example, about 0.01% to about 0.1% (w/w) or about 0.05% w/w;
   ii) about 0.05% (w/w) to about 5% (w/w) ginseng extract, or one or more ginsenosides, for example, about 0.5% to about 2% (w/w) or about 1% (w/w);
   iii) about 0.05% (w/w) to about 5% (w/w) notoginseng extract, or one or more notoginsenosides, for example, about 0.5% to about 2% (w/w) or about 1% (w/w);
   iv) about 0.01% to about 5% (w/w) zinc ion source, for example, about 0.1% to about 1% (w/w) or about 0.2% (w/w), for example, zinc citrate and/or zinc oxide;
   v) about 0.01% to about 5% (w/w) hyaluronic acid, for example, about 0.1% to about 1% (w/w) or about 0.3% (w/w);
   vi) about 0.01% to about 5% (w/w) pectin, for example, about 0.1% to about 1% (w/w) or about 0.3% (w/w); and
   vii) about 0.05% to about 5% (w/w) quercetin, for example, about 0.5% to about 2% (w/w) or about 1% (w/w); and
d) one or more inactive carriers.

According to a further aspect of the invention, there is provided an oral care composition, for example, a tooth whitening toothpaste, comprising:
a) about 0.1% to about 10% (w/w) of a compound of formula (I) as defined herein, for example, about 0.5% ot about 2% (w/w) or about 1% (w/w) for example, urolithin A; and
b) about 1% to about 40% (w/w) of whitening agent, such as a peroxide source, for example, hydrogen peroxide, for example, about 3% to about 10% (w/w) or about 5% (w/w); and
c) one or more inactive carriers.

According to a further aspect of the invention, there is provided an oral care composition, for example, a tooth whitening toothpaste, comprising:
a) about 0.1% to about 10% (w/w) of a compound of formula (I) as defined herein, for example, about 0.5% ot about 2% (w/w) or about 1% (w/w) for example, urolithin A;
b) about 1% to about 40% (w/w) hydrogen peroxide, for example, about 3% to about 10% (w/w) or about 5% (w/w); and
c) one or more of the following components:
   i) about 0.005% to about 1% (w/w) PQQ, for example, about 0.01% to about 0.1% (w/w) or about 0.05% w/w;
   ii) about 25 to about 25,000 ppm of a fluoride source, for example, about 500 to about 5000 ppm or about 1500 ppm, for example, sodium fluoride;
   iii) about 1% to about 50% (w/w) hydroxyapatite, for example, about 5% to about 20% (w/w) or about 10% (w/w);
   iv) about 0.05% (w/w) to about 5% (w/w) ginseng extract, or one or more ginsenosides, for example, about 0.5% to about 2% (w/w) or about 1% (w/w);
   v) about 0.05% (w/w) to about 5% (w/w) notoginseng extract, or one or more notoginsenosides, for example, about 0.5% to about 2% (w/w) or about 1% (w/w);
   vi) about 0.01% to about 5% (w/w) zinc ion source, for example, about 0.1% to about 1% (w/w) or about 0.2% (w/w), for example, zinc citrate and/or zinc oxide; vii) about 0.01% to about 5% (w/w) hyaluronic acid, for example, about 0.1% to about 1% (w/w) or about 0.3% (w/w);
   viii) about 0.01% to about 5% (w/w) pectin, for example, about 0.1% to about 1% (w/w) or about 0.3% (w/w);
   ix) about 0.05% to about 5% (w/w) quercetin, for example, about 0.5% to about 2% (w/w) or about 1% (w/w); and
d) one or more inactive carriers.

According to a further aspect of the invention, there is provided an oral care composition, for example, a tooth whitening toothpaste, comprising:
a) about 0.1% to about 10% (w/w) of a compound of formula (I) as defined herein, for example, about 0.5% ot about 2% (w/w) or about 1% (w/w) for example, urolithin A;
b) about 1% to about 40% (w/w) hydrogen peroxide, for example, about 3% to about 10% (w/w) or about 5% (w/w);
c) about 25 to about 25,000 ppm of a fluoride source, for example, about 500 to about 5000 ppm or about 1500 ppm, for example, sodium fluoride; and
d) one or more of the following components:
   i) about 0.005% to about 1% (w/w) PQQ, for example, about 0.01% to about 0.1% (w/w) or about 0.05% w/w;
   ii) about 0.05% (w/w) to about 5% (w/w) ginseng extract, or one or more ginsenosides, for example, about 0.5% to about 2% (w/w) or about 1% (w/w);
   iii) about 0.05% (w/w) to about 5% (w/w) notoginseng extract, or one or more notoginsenosides, for example, about 0.5% to about 2% (w/w) or about 1% (w/w);
   iv) about 0.01% to about 5% (w/w) zinc ion source, for example, about 0.1% to about 1% (w/w) or about 0.2% (w/w), for example, zinc citrate and/or zinc oxide; v) about 0.01% to about 5% (w/w) hyaluronic acid, for example, about 0.1% to about 1% (w/w) or about 0.3% (w/w);
   vi) about 0.01% to about 5% (w/w) pectin, for example, about 0.1% to about 1% (w/w) or about 0.3% (w/w);
   vii) about 0.05% to about 5% (w/w) quercetin, for example, about 0.5% to about 2% (w/w) or about 1% (w/w); and
e) one or more inactive carriers.

According to a further aspect of the invention, there is provided an oral care composition, for example, a tooth whitening toothpaste, comprising:
a) about 0.1% to about 10% (w/w) of a compound of formula (I) as defined herein, for example, about 0.5% ot about 2% (w/w) or about 1% (w/w) for example, urolithin A;
b) about 1% to about 50% (w/w) of a fluoride replacement, for example, hydroxyapatite, for example, about 5% to about 20% (w/w) or about 10% (w/w);
c) about 1% to about 40% (w/w) hydrogen peroxide, for example, about 3% to about 10% (w/w) or about 5% (w/w); and
d) one or more of the following components:
   a. about 0.005% to about 1% (w/w) PQQ, for example, about 0.01% to about 0.1% (w/w) or about 0.05% w/w;
   b. about 0.05% (w/w) to about 5% (w/w) ginseng extract, or one or more ginsenosides, for example, about 0.5% to about 2% (w/w) or about 1% (w/w);
   c. about 0.05% (w/w) to about 5% (w/w) notoginseng extract, or one or more notoginsenosides, for example, about 0.5% to about 2% (w/w) or about 1% (w/w);
   d. about 0.01% to about 5% (w/w) zinc ion source, for example, about 0.1% to about 1% (w/w) or about 0.2% (w/w), for example, zinc citrate and/or zinc oxide;
   e. about 0.01% to about 5% (w/w) hyaluronic acid, for example, about 0.1% to about 1% (w/w) or about 0.3% (w/w);
   f. about 0.01% to about 5% (w/w) pectin, for example, about 0.1% to about 1% (w/w) or about 0.3% (w/w);
   g. about 0.05% to about 5% (w/w) quercetin, for example, about 0.5% to about 2% (w/w) or about 1% (w/w); and
e) one or more inactive carriers.

According to a further aspect of the invention, there is provided an oral care composition, for example, a mouthwash, comprising:
a) about 0.1% to about 10% (w/w) of a compound of formula (I) as defined herein, for example, about 0.5% ot about 2% (w/w) or about 1% (w/w) for example, urolithin A; and
b) about 0.05% to about 2% menthol, for example, about 0.1% to about 1% (w/w) or about 0.4% (w/w),
c) one or more inactive carriers.

According to a further aspect of the invention, there is provided an oral care composition, for example, a mouthwash, comprising:
a) about 0.1% to about 10% (w/w) of a compound of formula (I) as defined herein, for example, about 0.5% to about 2% (w/w) or about 1% (w/w) for example, urolithin A;
b) about 0.05% to about 2% menthol, for example, about 0.1% to about 1% (w/w) or about 0.4% (w/w); and
c) one or more of the following components:
   i) about 0.005% to about 1% (w/w) PQQ, for example, about 0.01% to about 0.1% (w/w) or about 0.05% w/w;
   ii) about 0.05% (w/w) to about 5% (w/w) ginseng extract, or one or more ginsenosides, for example, about 0.5% to about 2% (w/w) or about 1% (w/w);
   iii) about 0.05% (w/w) to about 5% (w/w) notoginseng extract, or one or more notoginsenosides, for example, about 0.5% to about 2% (w/w) or about 1% (w/w);
   iv) about 0.01% to about 5% (w/w) zinc ion source, for example, about 0.1% to about 1% (w/w) or about 0.2% (w/w), for example, zinc citrate or zinc oxide;
   v) about 0.01% to about 5% (w/w) Arabic gum, for example, about 0.1% to about 1% (w/w) or about 2% (w/w);
   vi) about 0.05% to about 5% (w/w) quercetin, for example, about 0.5% to about 2% (w/w) or about 1% (w/w); and
d) one or more inactive carriers.

### Urolithins

Urolithins are metabolites produced by the action of mammalian, including human, gut microbiota on ellagitannins and ellagic acid. Ellagitannins and ellagic acid are compounds commonly found in foods such as pomegranates, nuts and berries. Ellagitannins are minimally absorbed in the gut themselves. Urolithins are a class of compounds with the representative structure (I) shown below. The structures of some particularly common urolithins are described in Table 1 below, with reference to structure (I).

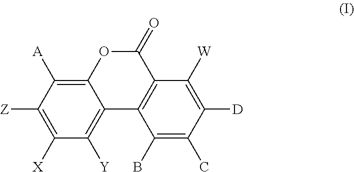

| | Substituent of structure (I) | | | | | |
|---|---|---|---|---|---|---|
| | A | B | C | D | W, X and Y | Z |
| Urolithin A | H | H | H | OH | H | OH |
| Urolithin B | H | H | H | H | H | OH |
| Urolithin C | H | H | OH | OH | H | OH |
| Urolithin D | OH | H | OH | OH | H | OH |
| Urolithin E | OH | OH | H | OH | H | OH |
| Isourolithin A | H | H | OH | H | H | OH |
| Isourolithin B | H | H | OH | H | H | H |
| Urolithin M-5 | OH | OH | OH | OH | H | OH |
| Urolithin M-6 | H | OH | OH | OH | H | OH |
| Urolithin M-7 | H | OH | H | OH | H | OH |

In practice, for commercial scale products, it is convenient to synthesise the urolithins. Routes of synthesis are described, for example, in WO 2014/004902, WO 2015/100213 and WO 2019/168972.

Urolithins of any structure according to structure (1) may be used in the compositions of the invention.

In one aspect of a compositions of the invention, a suitable compound is a compound of formula (I) wherein A, C, D and Z are independently selected from H and OH and B, W, X and Y are all H, preferably at least one of A, C, D and Z is OH.

Particularly suitable compounds are the naturally-occurring urolithins. Thus, Z is preferably OH and W, X and Y are preferably all H. When W, X and Y are all H, and A, and B are both H, and C, D and Z are all OH, then the compound is Urolithin C. When W, X and Y are all H, and A, B and C are all H, and D and Z are both OH, then the compound is urolithin A. Preferably, the urolithin used in the methods of the present disclosure is urolithin A, urolithin B, urolithin C or urolithin D. Most preferably, the urolithin used is urolithin A.

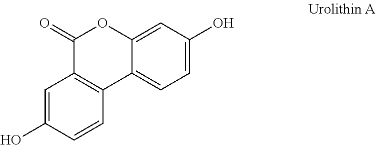 [SMILES: O=c1oc2cc(O)ccc2c2ccc(O)cc12]

According to one embodiment there is provided a composition of the invention wherein the compound of formula (I) is urolithin A.

According to one embodiment there is provided a composition of the invention wherein the compound of formula (I) is urolithin B.

According to one embodiment there is provided a composition of the invention wherein the compound of formula (I) is urolithin C.

According to one embodiment there is provided a composition of the invention wherein the compound of formula (I) is urolithin D.

In one embodiment, urolithins comprise acylated urolithins or optionally substituted acylated urolithins, (for example, acylated urolithin A, acylated urolithin B, acylated urolithin C, acylated urolithin D, acylated urolithin E, or acylated urolithin M5; or urolithin C having at least one hydroxyl substituted with a group containing a fatty acid). The term “acyl,” as used herein, represents a chemical substituent of formula —C(O)—R, where R is alkyl, alkenyl, aryl, arylalkyl, cycloalkyl, heterocyclyl, heterocyclyl alkyl, heteroaryl, or heteroaryl alkyl. An optionally substituted acyl is an acyl that is optionally substituted as described herein for each group R. Examples of acyl include fatty acid acyls (e.g., short chain fatty acid acyls (e.g., acetyl)) and benzoyl. In once embodiment, the acylated urolithin is a urolithin substituted by one or more C₁₋₄alkylcarbonyl groups, more example, urolithin A substituted by one or two C₁₋₄alkylcarbonyl groups (for example, formyl, acetyl or propionyl), for example, urolithin A acetate or urolithin A diacetate.

The present invention also encompasses use of suitable salts of compounds of formula (I), e.g. pharmaceutically acceptable salts. Suitable salts according to the invention include those formed with organic or inorganic bases. Pharmaceutically acceptable base salts include ammonium salts, alkali metal salts, for example those of potassium and sodium, alkaline earth metal salts, for example those of calcium and magnesium, and salts with organic bases, for example dicyclohexylamine, N-methyl-D-glucomine, morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl-propylamine, or a mono-, di- or trihydroxy lower alkylamine, for example mono-, di- or triethanolamine.

### Ginsenq Extracts and Qinsenosides

Ginseng is an ancient perennial herb belonging to the family Araliaceae and genus Panax (Panax ginseng) which has been used for medical therapeutics for thousands of years, particularly in China and other Asian cultures. In the last three decades, it has become one of the most popular herbs worldwide. It is used in agricultural products, dietary and health supplements, and medicines in different countries. The dried roots and rhizomes of ginseng contain many physiologically important constituents. These include ginseng saponins, ginseng oils and phytosterol, carbohydrates and sugars, organic acids, nitrogenous substances, amino acids and peptides, vitamins and minerals, and certain enzymes that have been isolated and characterized. The saponins are referred to as ginsenosides and are the most active constituents. Although the effects of ginseng are not solely dependent on ginsenosides, and other active ingredients have been described, such as gintonins.

Generally, a ginseng extract may be prepared from ginseng leaves and/or roots (preferably roots), in a variety of ways known in the art (see, for example, Oshima et al. J. Nat. Prod., March-April 1987, pp. 188-190; Shoji, J. Chemistry of Ginseng; in Yakuyoninjin Recent-Studies on Ginseng, Tokyo, Kyoritsu Publishing Co., 1981, p. 10), and normally yields 10-40% by weight of the ginseng plant. Methods for preparing ginseng extracts are also disclosed in International application, WO 1999/030725 (PCT/US98/25724).

Ginseng extracts may contain so-called primary ginsenosides (Rb1, Rb2, Rc, Rd, Re, Rg1, etc.) or secondary ginsenosides (Compound K, Rg3, Rh1, Rh2, F1, F2) derived from the transformation of primary ginsenosides.

These extracts include: white ginseng extracts (Panax ginseng), which primarily contain the ginsenosides Rb1, Rb2, Rc, and Rd; Red ginseng extract (Panax ginseng), which primarily contains the ginsenosides Rg1, Rg3, Rh1, Rh2, and Rf; and American ginseng extract (Panax quinquefolius), which primarily contains the dominant ginsenosides Re and Rb1. Ginseng leaf extract can also be used, which is rarer but contains significant quantities of ginsenosides (notably Rg1, Rb1). Finally, fermented Ginseng extracts in which the ginsenosides are transformed by fermentation leading to the generation of more bioavailable secondary forms (Compound K (metabolite of Rb1), Rh1, Rh2, Rg3, F1, F2) According to a further embodiment of the invention, there is provided a composition of the invention wherein the ginsenosides are one or more selected from Rb1, Rb2, Rg3, Rh2, Rh3, Rg1, Rg2, and Rh1.

According to a further embodiment of the invention, there is provided a composition of the invention comprising ginsenoside Rg1.

According to a further embodiment of the invention, there is provided a composition of the invention comprising ginsenoside Rg1 and ginsenoside Rb1.

According to a further embodiment of the invention, there is provided a composition of the invention comprising gintonins.

According to a further embodiment of the invention, there is provided a composition of the invention comprising a red ginseng extract, a white ginseng extract, an American ginseng or a mixture of an American ginseng extract, a red ginseng extract and/or a white ginseng extract.

### Notoginseng Extracts and Notoginsenoside

Notoginseng is a related perennial herb, also belonging to the family Araliaceae and genus Panax (Panax notoginseng) and is also widely used in traditional Chinese medicine. Notoginseng also contains saponins which are referred to as notoginsenosides, although some of the notoginsenosides are also found in ginseng and some are unique to notoginseng. A review of ginsenosides and notoginsenosides is Liu et al (2020) Pharmacological Research 161, 105263.

A number of ginsenosides and notoginsenosides have been identified. Some of these saponin are shared between ginseng and notoginseng, for example, Rb1, Rd, Re, Rg1, Rg2, Rh1, and gypenoside XVII. Therefore, according to a further aspect of the invention, there is provided a composition of the invention, wherein the ginsenosides and/or notoginsenosides comprise one or more selected from Rb1, Rd, Re, Rg1, Rg2, Rh1, and gypenoside XVII. Of these Rg1 and Rb1 are the most abundant and therefore, according to a further embodiment of the invention, there is provided a composition wherein the ginsenosides and/or notoginsenosides comprise one or more selected from Rg1 and Rb1.

Notoginseng also contains notoginsenosides which are different from ginsenoside compounds found in ginseng, these include: R1, Rt, R2, R3, R4, and R6. Therefore, according to a further embodiment of the invention, there is provided a composition of the invention wherein the notoginsenosides are one or more selected from R1, Rt, R2, R3, R4, and R6.

In a further embodiment of the invention, compositions of the invention comprise one or more agents to reduce plaque and reduce gingival inflammation. Examples of such agents include, ginseng extracts, ginsenosides and saponins, aloe vera gel, green tea extract, Epigallocatechin-3-gallate (EGCG), L-Theanine, curcumin, chamomile extract, myrrh extract, cinnamon extract, and/or hydroxytyrosol (for example, from olive leaf extract)

### Toothpaste

Oral compositions of the invention include toothpaste, which may optionally comprise additional ingredients suitable for use in oral care compositions. Examples of such ingredients include active agents, such as a phosphate source, for example, sodium phosphate and/or calcium phosphate. The compositions may be formulated in a suitable toothpaste base, e.g., comprising abrasives, e.g., silica abrasives, surfactants, foaming agents, vitamins, polymers, humectants, thickeners, additional antimicrobial agents, preservatives, flavourings, colourings, and/or combinations thereof. Examples of suitable toothpaste bases are known in the art. Alternatively, the compositions may be formulated as a gel (e.g., for use in a tray), chewing gum, lozenge or mint. Examples of suitable additional ingredients that can be employed in the compositions of the present disclosure are discussed in more detail below.

### Active Agents:

The compositions of the disclosure may comprise various other agents that are active to protect and enhance the strength and integrity of the enamel and tooth structure and/or to reduce bacteria and associated tooth decay and/or gum disease or to provide other desired benefits. Effective concentration of the active ingredients used herein will depend on the particular agent and the delivery system used. The concentration will also depend on the exact salt or polymer selected. For example, where the active agent is provided in salt form, the counterion will affect the weight of the salt, so that if the counterion is heavier, more salt by weight will be required to provide the same concentration of active ion in the final product.

Compositions of the disclosure may contain from 0.1 to 1 wt % of an antibacterial agent, such as about 0.3 wt. %. Any suitable antimicrobial actives can be employed.

In certain embodiments, oral care compositions comprise a fluoride source which includes sodium fluoride, sodium monofluorophosphate as well as mixtures thereof. In certain embodiments, the oral care composition of the disclosure may contain stannous fluoride and any additional source of fluoride ions or fluorine-providing agents in amounts sufficient to supply, in total, from 25 ppm to 25,000 ppm (mass fraction) of fluoride ions, generally at least 500 ppm, e.g. at least 1000 ppm, e.g., from 500 to 5000 ppm, e.g., from 500 to 2000 ppm, e.g. from 1000 to 2000 ppm, e.g., from about 1000 to about 1600 ppm, e.g., about 1500 or about 1600 ppm. The appropriate level of fluoride will depend on the particular application. A toothpaste for general consumer use would typically have from 1000 to about 1500 ppm, with paediatric toothpaste having somewhat less. A toothpaste or coating for professional application could have as much as 5,000 or even about 25,000 ppm fluoride. Additional fluoride ion sources may be added to the compositions of the disclosure at a level of from 0.01 wt. % to 10 wt. % in one embodiment or from 0.03 wt. % to 5 wt. %, and in another embodiment from 0.1 wt. % to 1 wt. % by weight of the composition. As discussed above, weights of fluoride salts to provide the appropriate level of fluoride ion will vary based on the weight of the counterion in the salt.

In other embodiments, oral compositions of the invention are essentially free of a fluoride source. Such oral compositions contain one of more ingredients to replace the protective effect of fluoride. Examples, include: arginine, xylitol, citric acid, zinc citrate, activated charcoal, calcium sodium phosphosilicate (Novamin), casein phosphopeptide-amorphous calcium phosphate (CPP-ACP), and hydroxyapatite, for example, calcium hydroxyapatite. In one embodiment, the fluoride alternative is hydroxyapatite. In a further embodiment, such ingredients include plant extracts such as extracts from papaya plant, Neem, Azadirachta indica, Tulsi, Ocimum sanctum, Prunus mume, Green and black tea (Camellia sinensi, Chinese Licorice Root, Salvadora persica, Prunusmume, and Hop plant (Humulus lupulus), and Oleic acid, Linoleic acid, epicatechin polymer (Cacao bean husk), proanthocyanidins, phenolic acids, flavonols (Cranberry), and Chlorophyll. In a further embodiments, such ingredient include propolis.

### Abrasives

The compositions of the disclosure can include abrasives. Examples of suitable abrasives include silica abrasives, such as standard cleaning silicas, high cleaning silicas or any other suitable abrasive silicas. Additional examples of abrasives that can be used in addition to or in place of the silica abrasives include, for example, a calcium phosphate abrasive, e.g., tricalcium phosphate (Ca₃(PO₄)₂), hydroxyapatite (Ca₁₀(PO₄)₆(OH)₂), or dicalcium phosphate dihydrate (CaHPO₄·2H₂0, also sometimes referred to herein as DiCal) or calcium pyrophosphate; calcium carbonate abrasive; or abrasives such as sodium metaphosphate, potassium metaphosphate, aluminium silicate, calcined alumina, bentonite or other siliceous materials, or combinations thereof.

Silica abrasive polishing materials useful herein, as well as the other abrasives, generally have an average particle size ranging between 0.1 and 30 microns, such as between 5 and 15 microns. The silica abrasives can be from precipitated silica or silica gels, such as the silica xerogels described in U.S. Pat. Nos. 3,538,230 and 3,862,307.

Particular silica xerogels are marketed under the trade name Syloid® by the W. R. Grace & Co., Davison Chemical Division. The precipitated silica materials include those marketed by the J. M. Huber Corp. under the trade name Zeodent®, including the silica carrying the designation Zeodent 115 and 119. These silica abrasives are described in U.S. Pat. No. 4,340,583, to Wason. In certain embodiments, abrasive materials useful in the practice of the oral care compositions in accordance with the disclosure include silica gels and precipitated amorphous silica having an oil absorption value of less than 100 cc/100 g silica, such as from 45 cc/100 g to 70 cc/100 g silica. Oil absorption values are measured using the ASTA Rub-Out Method D281. In certain embodiments, the silicas are colloidal particles having an average particle size of from 3 microns to 12 microns, and from 5 to 10 microns. Examples of low oil absorption silica abrasives useful in the practice of the disclosure are marketed under the trade designation Sylodent XWA® by Davison Chemical Division of W.R. Grace & Co., Baltimore, Md. 21203. Sylodent 650 XWA®, a silica hydrogel composed of particles of colloidal silica having a water content of 29% by weight averaging from 7 to 10 microns in diameter, and an oil absorption of less than 70 cc/100 g of silica is an example of a low oil absorption silica abrasive useful in the practice of the present disclosure.

Any suitable amount of silica abrasive can be employed. Examples of suitable amounts include 10 wt. % or more dry weight of silica particles, such as from 15 wt. % to 30 wt % or from 15 wt. % to 25 wt. %, based on the total weight of the composition.

### Surfactants:

The compositions useful in the compositions of the present disclosure may contain anionic surfactants, for example: (i). water-soluble salts of higher fatty acid monoglyceride monosulphates, such as the sodium salt of the monosulphated monoglyceride of hydrogenated coconut oil fatty acids such as sodium N-methyl N-cocoyl taurate, sodium cocomono glyceride sulphate,
(ii). higher alkyl sulphates, such as sodium lauryl sulphate,
(iii). higher alkyl-ether sulphates, e.g., of formula CH₃(CH₂)ₘCH₂(OCH₂CH₂)ₙOSO₃X, wherein m is 6-16, e.g., 10, n is 1-6, e.g., 2, 3 or 4, and X is Na or K, for example sodium laureth-2 sulphate (CH₃(CH₂)₁₀CH₂(OCH₂CH₂)₂OSO₃Na),
(iv). higher alkyl aryl sulphonates such as sodium dodecyl benzene sulphonate (sodium lauryl benzene sulphonate),
(v). higher alkyl sulphoacetates, such as sodium lauryl sulphoacetate (dodecyl sodium sulphoacetate), higher fatty acid esters of 1,2 dihydroxy propane sulphonate, sulphocolaurate (N-2-ethyl laurate potassium sulphoacetamide) and sodium lauryl sarcosinate.

In certain embodiments, the anionic surfactants useful herein include the water-soluble salts of alkyl sulphates having from 10 to 18 carbon atoms in the alkyl radical and the water-soluble salts of sulphonated monoglycerides of fatty acids having from 10 to 18 carbon atoms. Sodium lauryl sulphate, sodium lauroyl sarcosinate and sodium coconut monoglyceride sulphonates are examples of anionic surfactants of this type. In particular embodiments, the anionic surfactant is selected from sodium lauryl sulphate and sodium ether lauryl sulphate. In a particular embodiment, the compositions of the disclosure comprise sodium lauryl sulphate. The anionic surfactant may be present in an amount which is effective, e.g., >0.01% by weight of the formulation, but not at a concentration which would be irritating to the oral tissue, e.g., <10%, and optimal concentrations depend on the particular formulation and the particular surfactant. In one embodiment, the anionic surfactant is present in a toothpaste at from 0.3% to 4.5% by weight, e.g., about 1.5%. The compositions of the disclosure may optionally contain mixtures of surfactants, e.g., comprising anionic surfactants and other surfactants that may be anionic, cationic, zwitterionic or non-ionic. Generally, suitable surfactants are those which are reasonably stable throughout a wide pH range. Surfactants are described more fully, for example, in U.S. Pat. Nos. 3,959,458, 3,937,807, and 4,051,234.

The surfactant or mixtures of compatible surfactants that are included in addition to the anionic surfactants can be present in the compositions of the present disclosure in from 0.1% to 5.0%, in another embodiment from 0.3% to 3.0% and in another embodiment from 0.5% to 2.0% by weight of the total composition. These ranges do not include the anionic surfactant amounts.

In some embodiments, the compositions of the present disclosure include a zwitterionic surfactant, for example a betaine surfactant, for example cocamidopropylbetaine, e.g. in an amount of from 0.1% to 4.5% by weight, e.g. from 0.5 to 2% by weight cocamidopropylbetaine.

### Tartar Control Agents:

In various embodiments of the present disclosure, the compositions comprise an anti-calculus (tartar control) agent. Suitable anti-calculus agents include, without limitation, phosphates and polyphosphates (for example pyrophosphates and tripolyphosphates), polyaminopropanesulphonic acid (AMPS), hexametaphosphate salts, polyolefin sulphonates, polyolefin phosphates, and diphosphonates. The compositions of the disclosure thus may comprise phosphate salts in addition to the zinc phosphate. In particular embodiments, these salts are alkali phosphate salts, e.g., salts of alkali metal hydroxides or alkaline earth hydroxides, for example, sodium, potassium or calcium salts. In particular examples, the selected phosphate is selected from alkali dibasic phosphate and alkali pyrophosphate salts, e.g., selected from sodium phosphate dibasic, potassium phosphate dibasic, dicalcium phosphate dihydrate, calcium pyrophosphate, tetrasodium pyrophosphate, tetrapotassium pyrophosphate, sodium tripolyphosphate, and mixtures of any of two or more of these. In a particular embodiment, for example the compositions may comprise tetrasodium pyrophosphate in an amount of from 0.5 to 5% by weight, e.g., 1-3%, or 1-2% or about 2% by weight of the composition. In another embodiment, the compositions may comprise a mixture of tetrasodium pyrophosphate (TSPP) and sodium tripolyphosphate (STPP), e.g., in proportions of TSPP at from 0.5 to 5 wt. %, such as from 1 to 2 wt. % and STPP at from 0.5% to 6 wt. %, such as 1 to 4%, or 2 to 3% by weight of the composition. Such phosphates are provided in an amount effective to reduce erosion of the enamel, to aid in cleaning the teeth, and/or to reduce tartar buildup on the teeth, for example in an amount of from 0.2 to 20 wt. %, e.g., from 1 to 15 wt. %, by weight of the composition.

### Flavouring Agents:

The oral care compositions of the disclosure may also include a flavouring agent. Flavouring agents which are used in the practice of the present disclosure include, but are not limited to, essential oils as well as various flavouring aldehydes, esters, alcohols, and similar materials. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Certain embodiments employ the oils of peppermint and spearmint. The flavouring agent may be incorporated in the oral composition at a concentration of from 0.1 to 5% by weight e.g., from 0.5 to 1.5% by weight.

### Polymers

The oral care compositions of the disclosure may also include additional polymers to adjust the viscosity of the formulation or enhance the solubility of other ingredients. Such additional polymers include polysaccharides (e.g., cellulose derivatives, for example carboxymethyl cellulose, hydroxymethyl cellulose, ethyl cellulose, microcrystalline cellulose or polysaccharide gums, for example xanthan gum, guar gum or carrageenan gum). Acidic polymers, for example polyacrylate gels, may be provided in the form of their free acids or partially or fully neutralized water-soluble alkali metal (e.g., potassium and sodium) or ammonium salts. In one embodiment, the oral care composition may contain PVP.

Silica thickeners, which form polymeric structures or gels in aqueous media, may be present. Note that these silica thickeners are physically and functionally distinct from the particulate silica abrasives also present in the compositions, as the silica thickeners are very finely divided and provide little or no abrasive action. Other thickening agents are carboxyvinyl polymers, carrageenan, hydroxyethyl cellulose and water-soluble salts of cellulose ethers such as sodium carboxymethyl cellulose and sodium carboxymethyl hydroxyethyl cellulose. Natural gums such as karaya, gum arabic, and gum tragacanth can also be incorporated. Colloidal magnesium aluminium silicate can also be used as component of the thickening composition to further improve the composition's texture. In certain embodiments, thickening agents in an amount of from 0.1% to 5.0% by weight of the total composition are used.

In some embodiments, the compositions of the disclosure may include an anionic polymer, for example in an amount of from 0.05 to 5%. Examples of such agents generally known for use in toothpaste are disclosed in U.S. Pat. Nos. 5,188,821 and 5,192,531, and include synthetic anionic polymeric polycarboxylates, such as 1:4 to 4:1 copolymers of maleic anhydride or acid with another polymerizable ethylenically unsaturated monomer, preferably methyl vinyl ether/maleic anhydride having a molecular weight (M.W.) of from 30,000 to 1,000,000, such as from 300,000 to 800,000. These copolymers are available for example as Gantrez, e.g., AN 139 (M.W. 500,000), AN 119 (M.W. 250,000) and preferably S-97 Pharmaceutical Grade (M.W. 700,000) available from ISP Technologies, Inc., Bound Brook, N.J. 08805. The enhancing agents when present are present in amounts ranging from 0.05 to 3% by weight. Other operative polymers include those such as the 1:1 copolymers of maleic anhydride with ethyl acrylate, hydroxyethyl methacrylate, N-vinyl-2-pyrollidone, or ethylene, the latter being available for example as Monsanto EMA No. 1103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone. Suitable generally, are polymerized olefinically or ethylenically unsaturated carboxylic acids containing an activated carbon-to-carbon olefinic double bond and at least one carboxyl group, that is, an acid containing an olefinic double bond which readily functions in polymerization because of its presence in the monomer molecule either in the alpha-beta position with respect to a carboxyl group or as part of a terminal methylene grouping. Illustrative of such acids are acrylic, methacrylic, ethacrylic, alpha-chloroacrylic, crotonic, beta-acryloxy propionic, sorbic, alpha-chlorsorbic, cinnamic, beta-styrylacrylic, muconic, itaconic, citraconic, mesaconic, glutaconic, aconitic, alpha-phenylacrylic, 2-benzyl acrylic, 2-cyclohexylacrylic, angelic, umbellic, fumaric, maleic acids and anhydrides. Other different olefinic monomers co-polymerizable with such carboxylic monomers include vinylacetate, vinyl chloride, dimethyl maleate and the like. Copolymers contain sufficient carboxylic salt groups for water-solubility. A further class of polymeric agents includes a composition containing homopolymers of substituted acrylamides and/or homopolymers of unsaturated sulphonic acids and salts thereof, in particular where polymers are based on unsaturated sulphonic acids selected from acrylamidoalykane sulphonic acids such as 2-acrylamide 2 methylpropane sulphonic acid having a molecular weight of from 1,000 to 2,000,000. Another useful class of polymeric agents includes polyamino acids containing proportions of anionic surface-active amino acids such as aspartic acid, glutamic acid and phosphoserine, e.g. as disclosed in U.S. Pat. No. 4,866,161.

In some embodiments, there are no anionic polymers present in the composition. In other embodiments, there may be anionic polymers present, but they do not include copolymers of methyl vinyl ether and maleic acid or anhydride.

### Humectants

Within certain embodiments of the oral compositions, it is also desirable to incorporate a humectant to prevent the composition from hardening upon exposure to air. Certain humectants can also impart desirable sweetness or flavour to toothpaste compositions. Suitable humectants include edible polyhydric alcohols such as glycerine, sorbitol, xylitol, propylene glycol as well as other polyols and mixtures of these humectants. In one embodiment of the disclosure, the principal humectant is one of glycerine, sorbitol or a combination thereof. The humectant may be present at levels of greater than 15 wt. %, such as from 15 wt. % to 60 wt. %, or from 20 wt. % to 50 wt. %, or from 20 wt. % to 40 wt. %, or about 20% or about 30% or about 40%, based on the total weight of the composition.

### Other Optional Ingredients

In addition to the above-described components, the embodiments of this disclosure can contain a variety of optional oral care ingredients some of which are described below. Optional ingredients include, for example, but are not limited to adhesives, sudsing agents, flavouring agents, sweetening agents such as sodium saccharin, additional antiplaque agents, abrasives, aesthetics such as TiO₂ coated mica or other colouring agents, such as dyes and/or pigments.

In some embodiments, the compositions of the present disclosure can have any pH suitable for in a product for use in oral care. Examples of suitable pH ranges are from 6 to 9, such as from 6.5 to 8, or 6.5 to 7.5, or about 7.0.

In some embodiments, the oral care compositions of the present disclosure are either essentially free of, free of, or do not include any sodium hexametaphosphate. In some embodiments, the oral care compositions of the present disclosure are either essentially free of, free of, or do not include any halogenated diphenyl ethers (e.g., triclosan).

In some embodiments, the compositions of the present disclosure are either essentially free of, free of or do not include any complexing agents for increasing solubility of zinc phosphate and/or for maintaining the stannous fluoride in solution. Examples of known complexing agents that can be excluded from the compositions of the present disclosure include the chelating agents taught in U.S. Patent Application No. 2007/0025928. Such chelating agents include mineral surface-active agents, including mineral surface-active agents that are polymeric and/or polyelectrolytes and that are selected from phosphorylated polymers, wherein if the phosphorylated polymer is a polyphosphate, the polyphosphate has average chain length of 3.5 or more, such as 4 or more; polyphosphonates; polycarboxylates; carboxy-substituted polymers; copolymers of phosphate- or phosphonate-containing monomers or polymers with ethylenically unsaturated monomers, amino acids, proteins, polypeptides, polysaccharides, poly(acrylate), poly(acrylamide), poly(methacrylate), poly(ethacrylate), poly(hydroxyalkylmethacrylate), poly(vinyl alcohol), poly(maleic anhydride), poly(maleate) poly(amide), poly(ethylene amine), poly(ethylene glycol), poly(propylene glycol), poly(vinyl acetate) and poly(vinyl benzyl chloride); and mixtures thereof. Other known complexing agents that can be excluded from the compositions of the present disclosure include those taught in Canadian patent application, CA 2634758. Examples include polyphosphorylated inositol compounds such as phytic acid, myo-inositol pentakis(dihydrogen phosphate); myo-inositol tetrakis(dihydrogen phosphate), myo-inositol trikis(dihydrogen phosphate), and alkali metal, alkaline earth metal or ammonium salts of any of the above inositol compounds. Phytic acid is also known as myo-inositol 1,2,3,4,5,6-hexakis (dihydrogen phosphate) or inositol hexaphosphoric acid.

### Mouthwashes

In some embodiments, the oral care composition is not intentionally swallowed but rather retained in the oral cavity for a time sufficient to affect the intended utility. In some embodiments, the oral care composition is a mouthwash. A mouthwash of the invention may optionally comprise additional ingredients suitable for use in mouthwash composition. Examples of such ingredients include anti-bacterial agents, humectants, basic amino acids, a zinc ion source, preservatives, sweeteners, flavouring agents, pH modifying agents, buffering agents, anti-calculus agents, surfactants, polymers and anti-oxidants. For more information on the mouth components, the reader is referred to Radzki et al (2022) International Journal of Environmental Research & Public Health 19, 3926.

### Anti-Bacterial Agents

The oral composition of the present invention, for example, mouthwashes may include one or more anti-bacterial agents. Anti-bacterial agents include pyridinium compounds (e.g. cetylpyridinium chloride (CPC)), halogenated diphenyl ether (e.g. triclosan), herbal extracts and essential oils (e.g., rosemary extract, tea extract, magnolia extract, thymol, menthol, eucalyptol, geraniol, carvacrol, citral, hinokitol, catechol, methyl salicylate, epigallocatechin gallate, epigallocatechin, gallic acid, miswak extract, sea-buckthorn extract), bisguanide antiseptics (e.g., chlorhexidine, alexidine or octenidine), benzalkonium chloride, tetradecylpyridinium chloride (TPC), N-tetradecyl-4-ethylpyridinium chloride (TDEPC)), phenolic antiseptics, hexetidine, octenidine, sanguinarine, povidone iodine, delmopinol, salifluor, metal ions (e.g., zinc salts, for example, zinc chloride, zinc lactate, zinc sulphate, stannous salts, copper salts, iron salts), sanguinarine, propolis and oxygenating agents (e.g., hydrogen peroxide, buffered sodium peroxyborate or peroxycarbonate), phthalic acid and its salts, monoperthalic acid and its salts and esters, ascorbyl stearate, oleoyl sarcosine, alkyl sulphate, dioctyl sulphosuccinate, salicylanilide, domiphen bromide, delmopinol, octapinol and other piperidino derivatives, nicin preparations, chlorite salts; and mixtures of any of the foregoing.

### Humectants

The mouthwash composition of the present invention may include one or more humectants. Humectants can reduce evaporation and also contribute towards preservation by lowering water activity and can also impart desirable sweetness or flavour to compositions. Illustrative humectants may be or include, but are not limited to, glycerine, propylene glycol, sorbitol, xylitol, or the like, or any mixture or combination thereof. In a preferred embodiment, the orally acceptable vehicle may be or include, but is not limited to, glycerine or sorbitol. In some embodiments, the humectant is selected from glycerine, sorbitol and a combination thereof.

### Basic Amino Acids

The mouthwash composition of the present invention may include one or more basic amino acids in free or salt form. The basic amino acids which can be used in the compositions include not only naturally occurring basic amino acids, such as arginine, lysine, and histidine, but also any basic amino acids having a carboxyl group and an amino group in the molecule, which are water-soluble and provide an aqueous solution with a pH of about 7 or greater. Accordingly, basic amino acids include, but are not limited to, arginine, lysine, citrullene, ornithine, creatine, histidine, diaminobutanoic acid, di aminopropionic acid, salts thereof or combinations thereof. In a particular embodiment, the basic amino acids are selected from arginine, lysine, citrullene, and ornithine. The basic amino acids of the oral care composition may generally be present in the L-form or L-configuration. The basic amino acids may be provided as a salt of a di- or tri-peptide including the amino acid. In some embodiments, at least a portion of the basic amino acid present in the mouthwash composition is in the salt form. In some embodiments, the basic amino acid is arginine, for example, L-arginine, or a salt thereof. Arginine may be provided as free arginine or a salt thereof. For example, Arginine may be provided as arginine phosphate, arginine hydrochloride, arginine sulphate, arginine bicarbonate, or the like, and mixtures or combinations thereof. The basic amino acid may be provided as a solution or a solid. For example, the basic amino acid may be provided as an aqueous solution. In some embodiment, the amino acid includes or is provided by an arginine bicarbonate solution. For example, the amino acid may be provided by an about 40% solution of the basic amino acid, such as arginine bicarbonate or alternatively called as arginine carbamate.

### Zinc Ion Source

The mouthwash composition of the present invention may include a zinc ion source. The zinc ion source may be or include a zinc ion and/or one or more zinc salts. For example, the zinc salts may at least partially dissociate in an aqueous solution to produce zinc ions. Illustrative zinc salts may include, but are not limited to, zinc lactate, zinc oxide, zinc chloride, zinc phosphate, zinc citrate, zinc acetate, zinc borate, zinc butyrate, zinc carbonate, zinc formate, zinc gluconate, zinc glycerate, zinc glycolate, zinc picolinate, zinc proprionate, zinc salicylate, zinc silicate, zinc stearate, zinc tartrate, zinc undecylenate, and mixtures thereof. In some embodiments, the zinc ion source is selected from zinc oxide, zinc citrate, and a combination thereof. The avoidance of doubt other oral compositions of the invention, other than mouthwashes may comprise a zinc ion source.

### Preservatives

The mouthwash composition of the present invention may include a preservative. Suitable preservatives include, but are not limited to, sodium benzoate, potassium sorbate, methylisothiazolinone, paraben preservatives, for example methyl p-hydroxybenzoate, propyl p-hydroxybenzoate, and mixtures thereof. In certain embodiments, the preservative is sodium benzoate.

### pH Modifying Agents

The mouthwash composition of the present invention may include one or more pH modifying agents. For example, the oral care composition may include one or more acidifying agents and/or one or more basifying agents configured to reduce and/or increase the pH thereof, respectively. Illustrative acidifying agents and/or one or more basifying agents may be or include, but are not limited to, an alkali metal hydroxide, such as sodium hydroxide and/or potassium hydroxide, citric acid, hydrochloric acid, or the like, or combinations thereof.

### Buffering Agents

The mouthwash composition of the present invention may also include one or more buffering agents configured to control or modulate the pH within a predetermined or desired range. Illustrative buffering agents may include, but are not limited to, sodium bicarbonate, sodium phosphate, sodium carbonate, sodium acid pyrophosphate, sodium citrate, and mixtures thereof Sodium phosphate may include nionosodium phosphate (NaH2PO4), disodium phosphate (Na₂HPO₄), trisodium phosphate (Na₃PO₄), and mixtures thereof In a typical embodiment, the buffering agent may be anhydrous sodium phosphate dibasic or disodium phosphate and/or sodium phosphate monobasic. In another embodiment, the buffering agent includes anhydrous sodium phosphate dibasic or disodium phosphate, and phosphoric acid (e.g., syrupy phosphoric acid; 85%-Food Grade).

### Anti-Calculus Agents

The mouthwash composition of the present invention may include anti-calculus agents. Illustrative anti-calculus agents may include, but are not limited to, phosphates and polyphosphates (e.g., pyrophosphates), polyaminopropanesulphonic acid (AMPS), hexametaphosphate salts, zinc citrate trihydrate, polypeptides, polyolefin sulphonates, polyolefin phosphates, diphosphonates. In some embodiments, the anticalculus agent includes tetrasodium pyrophosphate (TSPP), sodium tripolyphosphate (STPP), or a combination thereof.

### Chelating Agents

The mouthwash composition of the present invention also may include one or more chelating agents able to complex calcium found in the cell walls of the bacteria. Binding of this calcium weakens the bacterial cell wall and augments bacterial lysis.

### Surfactants

The invention may in some embodiments contain anionic surfactants, for example, water-soluble salts of higher fatty acid monoglyceride monosulphates, such as the sodium salt of the monosulphated monoglyceride of hydrogenated coconut oil fatty acids such as sodium N-methyl N-cocoyl taurate, sodium cocomo-glyceride sulphate; higher alkyl sulphates, such as sodium lauryl sulphate; higher alkyl-ether sulphates, e.g., of formula CH₃(CH₂)ₘCH₂(OCH₂CH₂)nOS0₃X, wherein m is 6-16, e.g., 10, n is 1-6, e.g., 2, 3 or 4, and X is sodium or, for example sodium laureth-2 sulphate (CH₃(CH₂)₁₀CH₂(OCH₂CH₂)₂OSO₃Na); higher alkyl aryl sulphonates such as sodium dodecyl benzene sulphonate (sodium lauryl benzene sulphonate); higher alkyl sulphoacetates, such as sodium lauryl sulphoacetate (dodecyl sodium sulphoacetate), higher fatty acid esters of 1,2 dihydroxy propane sulphonate, sulphocolaurate (N-2-ethyl laurate potassium sulphoacetamide) and sodium lauryl sarcosinate. In particular embodiments, the anionic surfactant (where present) is selected from sodium lauryl sulphate and sodium ether lauryl sulphate. When present, the anionic surfactant is present in an amount which is effective, e.g., >0.001% by weight of the formulation, but not at a concentration which would be irritating to the oral tissue, e.g., 1%, and optimal concentrations depend on the particular formulation and the particular surfactant. In one embodiment, the anionic surfactant is present at from 0.03% to 5% by weight, e.g., 1.5%.

In another embodiment, cationic surfactants useful in the present invention can be broadly defined as derivatives of aliphatic quaternary ammonium compounds having one long alkyl chain containing 8 to 18 carbon atoms such as lauryl trimethylammonium chloride, cetyl pyridinium chloride, cetyl trimethylammonium bromide, di-isobutylphenoxyethyldimethylbenzylammonium chloride, coconut alkyltrimethylammonium nitrite, cetyl pyridinium fluoride, and mixtures thereof. Illustrative cationic surfactants are the quaternary ammonium fluorides described in U.S. Pat. No. 3,535,421. Certain cationic surfactants can also act as germicides in the compositions.

Non-ionic surfactants that can be used in the compositions of the invention can be broadly defined as compounds produced by the condensation of alkylene oxide groups (hydrophilic in nature) with an organic hydrophobic compound which may be aliphatic or alkylaromatic in nature. Examples of suitable non-ionic surfactants include, but are not limited to, the Pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulphoxides and mixtures of such materials. In a particular embodiment, the composition of the invention comprises a non-ionic surfactant selected from polaxamers (e.g., polaxamer 407), polysorbates (e.g., polysorbate 20), polyoxyl hydrogenated castor oils (e.g., polyoxyl 40 hydrogenated castor oil), and mixtures thereof.

In still another embodiment amphoteric surfactants can be used. Suitable amphoteric surfactants, without limitation, are derivatives of C₈₋₂₀aliphatic secondary and tertiary amines having an anionic group such as carboxylate, sulphate, sulphonate, phosphate or phosphonate. A suitable example is cocoamidopropyl betaine. One or more surfactants are optionally present in a total amount of 0.01 weight % to 10 weight %, for example, from 0.05 weight % to 5 weight % or from 0.1 weight % to 2 weight % by total weight of the composition.

The surfactant or mixtures of compatible surfactants can be present in the compositions of the present invention in 0.1% to 5%, in another embodiment 0.3% to 3% and in another embodiment 0.5% to 2% by weight of the total composition.

### Polymers

The mouthwash compositions of the invention also optionally include one or more polymers, such as polyethylene glycols, polyvinyl methyl ether maleic acid copolymers, polysaccharides (e.g., cellulose derivatives, for example carboxymethylcellulose, or polysaccharide gums, for example xanthan gum or carrageenan gum). Acidic polymers, for example polyacrylate gels, may be provided in the form of their free acids or partially or fully neutralized water soluble alkali metal (e.g., potassium and sodium) or ammonium salts. Certain embodiments include 1:4 to 4:1 copolymers of maleic anhydride or acid with another polymerizable ethylenically unsaturated monomer, for example, methyl vinyl ether (methoxyethylene) having a molecular weight (M.W.) of about 30,000 to about 1,000,000. These copolymers are available for example as Gantrez AN 139 (M.W. 500,000), AN 1 19 (M.W. 250,000) and S-97 Pharmaceutical Grade (M.W. 70,000), of GAF Chemicals Corporation.

Other operative polymers include those such as the 1:1 copolymers of maleic anhydride with ethyl acrylate, hydroxyethyl methacrylate, N-vinyl-2-pyrollidone, or ethylene, the latter being available for example as Monsanto EMA No. 1 103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone.

Suitable generally, are polymerized olefinically or ethylenically unsaturated carboxylic acids containing an activated carbon-to-carbon olefinic double bond and at least one carboxyl group, that is, an acid containing an olefinic double bond which readily functions in polymerization because of its presence in the monomer molecule either in the alpha-beta position with respect to a carboxyl group or as part of a terminal methylene grouping. Illustrative of such acids are acrylic, methacrylic, ethacrylic, alpha-chloroacrylic, crotonic, beta-acryloxy propionic, sorbic, alpha-chlorsorbic, cinnamic, beta-styrylacrylic, muconic, itaconic, citraconic, mesaconic, glutaconic, aconitic, alpha-phenylacrylic, 2-benzyl acrylic, 2-cyclohexylacrylic, angelic, umbellic, fumaric, maleic acids and anhydrides. Other different olefinic monomers copolymerizable with such carboxylic monomers include vinylacetate, vinyl chloride, dimethyl maleate and the like. Copolymers contain sufficient carboxylic salt groups for water-solubility.

A further class of polymeric agents includes a composition containing homopolymers of substituted acrylamides and/or homopolymers of unsaturated sulfonic acids and salts thereof, in particular, where polymers are based on unsaturated sulfonic acids selected from acrylamidoalykane sulfonic acids such as 2-acrylamide 2-methylpropane sulfonic acid having a molecular weight of about 1,000 to about 2,000,000, described in U.S. Pat. No. 4,842,847.

Another useful class of polymeric agents includes polyamino acids, particularly those containing proportions of anionic surface-active amino acids such as aspartic acid, glutamic acid and phosphoserine, as disclosed in U.S. Pat. No. 4,866,161.

### Enzymes

The mouthwash compositions of the disclosure may also optionally include one or more enzymes. Useful enzymes include any of the available proteases, glucanohydrolases, endoglycosidases, amylases, mutanases, lipases and mucinases or compatible mixtures thereof In certain embodiments, the enzyme is a protease, dextranase, endoglycosidase and mutanase. In another embodiment, the enzyme is papain, endoglycosidase or a mixture of dextranase and mutanase. Additional enzymes suitable for use in the present invention are disclosed in U.S. Pat. Nos. 5,000,939, 4,992,420; 4,355,022; 4,154,815; 4,058,595; 3,991,177; and 3,696,191. An enzyme of a mixture of several compatible enzymes in the current invention constitutes 0.002% to 2.0% in one embodiment or 0.05% to 1.5% in another embodiment or in yet another embodiment 0.1% to 0.5%.

### Sweeteners

The mouthwash composition of the present invention may include a sweetener such as, for example, saccharin, for example sodium saccharin, acesulpham, neotame, cyclamate or sucralose; natural high-intensity sweeteners such as allulose, thaumatin, stevioside, rebaudioside, or glycyrrhizin; or such as sorbitol, xylitol, maltitol or mannitol. In certain embodiments, the sweetener is saccharin, e.g., sodium saccharin. In another embodiment, the sweetener is rebaudioside m or allulose.

### Flavourings

The mouthwash composition of the present invention may include a flavouring agent. Suitable flavouring agents include, but are not limited to, essential oils and various flavouring aldehydes, esters, alcohols, and similar materials, as well as sweeteners such as sodium saccharin. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole.

### Dental Floss, Tape and Sticks (Picks)

In practice, floss normally would comprise a large number of filaments or strands. Suitable filaments may be in the form of a single strand or tape of any acceptable material such as nylon, polypropylene, polyester, silk, cotton, including cotton with a binding agent such as a modified nylon resin, vinyl acetate polymer, or Gantrez (polyvinyl methyl/ether/maleic anhydride copolymer). These materials usually are coated with a wax, which may be water soluble or insoluble (not shown). Acceptable waxes are, for example, microcrystalline hydrocarbon-based waxes, and silicone waxes.

A dental floss, tape or stick of the invention may optionally comprise additional ingredients suitable for use in such oral care products, such additional ingredients include a peroxide source, desensitizing agents, antimicrobial agents, tartar control agents, mouth feel agents, sweeteners, flavourings, colourings, and combinations thereof.

### a Peroxide Source

In one embodiment, the oral care product comprises a peroxide source. Exemplary peroxide compounds include hydroperoxides, hydrogen peroxide, organic peroxy compounds, peroxy acids, pharmaceutically acceptable salts thereof, and mixtures thereof. Organic peroxy compounds include urea peroxide, glyceryl hydrogen peroxide, alkyl hydrogen peroxides, dialkyl peroxides, alkyl peroxy acids, peroxy esters, diacyl peroxides, benzoyl peroxide, and mixtures thereof. Peroxy acids and their salts include organic peroxy acids such as alkyl peroxy acids and mixtures thereof, as well as inorganic peroxy acid salts such as and perborate salts of alkali and alkaline earth metals (e.g. lithium, potassium, sodium, magnesium, calcium and barium), and mixtures thereof Preferred solid peroxides are sodium perborate, sodium percarbonate, urea peroxide, and mixtures thereof In some embodiments, the peroxide compound may be bound to a polymer such as PVP (poly-N-vinylpyrrolidone. Suitable PVP complexes would be known to a person skilled in the art of oral care, and are also disclosed, for example, in U.S. Pat. No. 5,122,370. Linear PVP peroxide and cross-linked PVP peroxide sold as Peroxydone™ from ISP Corp. are also peroxide compounds useful. as whitening agents in the present invention. PVP-hydrogen peroxide can be effectively produced by mixing linear PVP or cross-linked PVP with concentrated liquid hydrogen peroxide in the oral care product.

Typically, the peroxide source comprises a peroxide compound selected from: urea peroxide, sodium percarbonate, sodium perborate. cross-linked PVP-hydrogen peroxide complex, and mixtures thereof. In a preferred embodiment, the peroxide source comprises cross-linked PVP-hydrogen peroxide. Cross-linked PVP-hydrogen peroxide has enhanced stability.

Typically, the peroxide source is present in the coating composition in an amount of from 0.1 weight % to 40 weight %, or from 1 weight % to 30 weight %, or from 5 weight % to 20 weight % by total weight of the composition. Preferably, the peroxide source is present in the composition in an amount of from 10 weight % to 20 weight %, or from 10 weight % to weight %, or from 8 weight % to 20 weight %, or from 8 weight % to 15 weight %, by total weight of the composition. In some embodiments, the peroxide source is present in the composition in an amount of from 10 weight % to 15 weight %, or from 1|weight % to 15 weight Rio by total weight of the composition, or from 0.12 weight % to 15 weight %, or from 13 weight % to 15 weight %, by total weight of the composition. Preferably, the peroxide source is present in the composition in an amount of from 10 weight % to 13 weight %, or from 10 weight % to 14 weight % by total weight of the composition. Typically, the peroxide source is present in the composition in an amount of 13 weight % by total weight of the composition. In the embodiments provided above, optionally, the peroxide source is cross-linked PVP-hydrogen peroxide

Preferably, the peroxide source is present in the composition in an amount sufficient to deliver hydrogen peroxide in an amount of from 0.1 weight % to 5 weight. %, or from 0.5 weight % to 5 weight %, or from 1 weight % to 5 weight %, or from 2 weight % to 5 weight %, or from 3 weight % to 5 weight % by total weight of the composition. In some embodiments, the peroxide source is present in the composition in an amount: sufficient to deliver hydrogen peroxide in an amount of from 0.25 weight % to 3 weight %, or from 0.5 weight % to 3 weight %, or from 1 weight % to 3 weight. %, or from 2 weight % to 3 weight %, by total weight of the composition. In further embodiments, the peroxide source is present in the composition in an amount sufficient to deliver hydrogen peroxide in an amount of from 0.5 weight % to 2 weight %, or from 1. weight % to 2 weight %, by total weight of the composition.

### Flavourings

The coating compositions provided herein may include a flavouring agent. Flavouring agents which are used in the practice of the present invention include, but are not limited to, essential oils and various flavouring aldehydes, esters, alcohols, and similar materials, as well as sweeteners such as sodium saccharin. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Certain embodiments employ the oils of peppermint and spearmint.

### Desensitizing Agents

The compositions provided herein optionally incorporate one or more desensitizing agents. These include, without limitation, potassium salts such as potassium nitrate, potassium bicarbonate, potassium chloride, potassium citrate, and potassium oxalate; capsaicin; eugenol; strontium salts; zinc salts; chloride salts and combinations thereof.

### Mouth-Feel Agents

Mouth-feel agents that may be incorporated into the compositions defined herein include materials which impart a desirable texture or other feeling during use of the floss. Such agents include bicarbonate salts, which may impart a “clean feel” to teeth. Any orally acceptable bicarbonate can be used, including, without limitation, alkali metal bicarbonates such as sodium and potassium bicarbonates, ammonium bicarbonate, and mixtures thereof.

### Sweeteners

The compositions provided herein may optionally comprise a sweetener. Sweeteners which may be used in the compositions of the present invention include artificial sweeteners such as saccharin, acesulfam, neotam, cyclamate or sucralose; natural high-intensity sweeteners such as thaumatin, stevioside or glycyrrhizin; or sugar alcohols such as sorbitol, xylitol, maltitol or mannitol.

### Colourings

The compositions provided herein may comprise at least one colorant. Colorants herein include pigments, dyes and agents imparting a particular luster or reflectivity such as pearling agents. Any orally acceptable colorant can be used, including without limitation talc, mica, magnesium carbonate, calcium carbonate, magnesium silicate, magnesium aluminum silicate, silica, titanium dioxide, zinc oxide, red, yellow, brown and black iron oxides, &irk ammonium ferrocyanide, manganese violet, ultramarine, titaniated mica, bismuth oxychloride and the like.

### Antimicrobial Agents

Preservatives, such as chlorhexidine, triclosan, quaternary ammonium compounds (such benzalkonium chloride) or parabens (such as methyl or propyl paraben) may be incorporated in the compositions provided herein. In a further embodiment, the composition comprises one or more of the other anti-bacterial agents disclosed herein.

### Thickening Agents

Thickening agents which may be incorporated into the compositions defined herein include natural and synthetic gums and colloids. Suitable thickening agents include naturally occurring polymers such as carrageenan, xanthan gum, polyglycols of varying molecular weights sold under the tradename Polyox, and polyvinylpyrrolidone (PVP). Compatible inorganic thickening agents include amorphous silica compounds and colloidal silica compounds available under the trade designation Zeodent manufactured by Huber Corporation. Other inorganic thickening agents include natural and synthetic clays such as hectorite clays, lithium magnesium silicate (laponite) and magnesium aluminum silicate (Veegum). PVP is a particularly preferred thickener since PVP retards the degradation of peroxide compounds such as PVP-peroxide. Additionally, coating compositions comprising PVP are more stable with less tendency for sedimentation. PVP may be present in the coating composition in an amount of from 1 to 5 weight %, from 1 weight % to 4 weight %, from 1 to 3 weight %, or from 1 weight % to 2 weight %, by total weight of the composition. Optionally, PVP is present in the coating composition in an amount of from 2 weight % to 4 weight, or from 2 weight % to 3 weight %, by total weight of the composition. In some embodiments, the PVP is present in the coating composition in an amount of 2 weight %, by total weight of the composition.

### Additional Components in Formulations of the Invention:

Oral care compositions of the invention may contain additional components beyond the compound of formula (I), for example, urolithin A. The additional components may be compounds that provide health benefits, for example, other bioactive compounds and/or mitochondria boosting agents.

In one embodiment, the bioactive compounds comprises betaine, bioflavonoids, resveratrol, coenzyme Q₁₀, quercetin, rutin, lycopene, spermidine, trigonelline, epicatechin, L-glutathione, N-acetyl cysteine, phenolics, anthocyanins, flavonoids, anthracenes, carotenoids, zeaxanthin, astaxanthin, xanthin, pomegranate, Ginkgo biloba, green tea, garlic, grapeseed, blackberry, elderberry, cranberry, blueberry, saffron, olive oil, for example, extra-virgin olive oil, Sangre de grado (dragon's blood), and lyceum barbarum (Gouqizi), its extract, powder, or isolates thereof.

In a further embodiment of the invention, there is provided a composition of the invention further comprising a senolytic and/or sensostatic compound. Example of senolytic compounds include: flavonoids and flavons such apigenin, galangin, rutin, myricetin, fisetin, Berberine, quercetin, and dihydroquercetin.

In a further embodiment, examples of senolytic compounds include dasatinib, quercetin, a mixture of dasatinib+quercetin, fisetin, piperlongumine, curcumin, EF-24, a mixture of curcumin and EF-24, Navitoclax (ABT-263), ABT-737, A1331852, A1155463, GL-V9 (wogonin derivative), procyanidin C1 (PCC1), fenofibrate, azithromycin, roxithromycin, and/or tamatinib (R406).

In a further embodiment, examples of senostatic compounds include resveratrol and SIRT-activating compounds (e.g. SRT1720, SRT2104), kaempferol, apigenin, EGCG, genistein, rutin, wogonin, metformin, JAK inhibitors (e.g. Ruxolitinib, Momelotinib), acetyl-salicylic acid and SR12343 (a IKK/NF-κB inhibitor),

In a further embodiment, bioactive agents include autophagy inducers and or mitochondrial biogenesis promoting agents.

Examples of autophagy inducers include, but not limited to, caffeine, omega-3 polyunsaturated fatty acids, spermidine, vitamin D pterostilbene, minoxidil, kinetin triphosphate, magnesium salts, D-ribose, and trehalose.

Further examples of autophagy inducers include, but not limited to, carbamazepine, clonidine, lithium, metformin, rapamycin (and rapalogs), rilmenidine, sodium valproate, verapamil, trifluoperazine, statins, tyrosine kinase inhibitors (for example, Akt-mTOR signaling inhibitors and beclin 1 tyrosine phosphorylation inhibitors), BH3 mimetics, actinonin, pifithrin-α, deferiprone and 1,10-phenanthroline.

Examples of mitochondrial biogenesis promoting agents include, but are not limited to, 1a axis activators (for example, bezafibrate), AMPK activators, Sirti agonists (for example, quercetin, resveratrol), anti-oxidants (such as L-carnitine, acetyl-L-carnitine, coenzyme Q₁₀, MitoQ₁₀ and other mitochondria-targeted antioxidants, N-acetylcysteine (NAC), vitamin C, vitamin E vitamin K1, vitamin B, sodium pyruvate and α-lipoic acid) and NAD precursors (for example, nicotinic acid (niacin), niacinamide (nicotinamide), trigonelline, tryptophan, nicotinamide mononucleotide and nicotinamide riboside).

### Medical and Non-Medical Treatments:

Compositions of the invention may be administered to subjects in need of therapeutic intervention, or to generally healthy subjects who would benefit from a composition of the invention. In particular, administration to subjects for disease and non-disease health conditions characterised by an inadequate mitochondrial activity. The compositions of the invention can be taken as a single dose or, more commonly, as a series of doses. In one example, a composition may, for example, be taken once, twice or three times per day, or one, two, three, four, five, six or seven times per week.

In a further embodiment there is provided a composition of the invention for use in a method of enhancing mitophagy, for example, in the oral cavity.

In a further embodiment there is provided a composition of the invention for use in a method of enhancing mitophagy in a generally healthy subject, for example, in the oral cavity.

In a further embodiment there is provided a composition of the invention for administration to a generally healthy subject who will benefit from an increase in mitophagy, for example, in the oral cavity.

In a further embodiment there is provided a composition of the invention for use in the treatment of a disease, disorder or condition associated with a decrease in mitophagy, for example, a disease, disorder or condition of the oral cavity.

In a further embodiment there is provided a composition of the invention for use in a method of enhancing or maintaining mitochondrial function, for example, in the oral cavity.

In a further embodiment there is provided a composition of the invention for use in the for treatment, prevention or management of a mitochondria-related condition associated with altered mitochondrial function or reduced mitochondrial density, for example, in the oral cavity.

In a further embodiment there is provided a composition of the invention for use in the treatment, prevention or management of a mitochondria-related condition associated with altered mitochondrial function or reduced mitochondrial density, for example, in the oral cavity.

In a further embodiment there is provided a composition of the invention for use in the treatment, prevention or management of a disease initiated or characterized by inadequate mitochondrial function.

In a further embodiment there is provided a composition of the invention for use in a method of increasing mitochondrial activity, increasing mitochondrial biogenesis, and/or increasing mitochondrial mass, for example, in the oral cavity.

In a further embodiment there is provided a composition of the invention for use in the treatment, prevention or management of diseases and conditions in which defective or diminished mitochondrial activity participates in the pathophysiology of the disease or condition, or in which increased mitochondrial function will yield a desired beneficial effect, for example, in the oral cavity.

In a further embodiment there is provided a composition of the invention for use in the treatment, prevention or management of a mitochondrial disease.

Compositions of the invention, for example, are effective upon application to the oral cavity, for example, by rinsing, optionally in conjunction with brushing, for example, one or more times per day.

In a further embodiment there is provided a composition of the invention for use in improving appearance and/or making structural changes to teeth, stain bleaching, stain removal, plaque removal, tartar removal, cavity prevention and treatment, inflamed and/or bleeding gums, mucosal wounds, lesions, ulcers, aphthous ulcers, cold sores and tooth abscesses, oral malodour, dental erosion, gingivitis, and/or periodontal disease.

In a further embodiment there is provided a composition of the invention for use treating in an oral disease, disorder, or condition characterised by inadequate mitochondrial activity.

### Non-Medical Treatments:

In a further embodiment, there is provided the use of a composition of the invention for maintaining or enhancing mitochondrial function, for example, in the oral cavity.

In a further embodiment there is provided the use of a composition of the invention in a method of increasing mitochondrial activity, increasing mitochondrial biogenesis, and/or increasing mitochondrial mass, for example, in the oral cavity.

In a further embodiment there is provided the use of a composition of the invention in a method of enhancing mitophagy, for example, in the oral cavity.

In a further embodiment, there is provided a non-therapeutic method of treating a condition selected from one or more of the following:
(a). enhancing oral healthspan;
(b). enhancing oral longevity;
(c). enhancing gum healthspan;
(d). enhancing gum longevity;
(e). enhancing tongue healthspan;
(f). enhancing tongue longevity′
(g). maintaining or enhancing oral muscosa health;
(h). maintaining or enhancing oral epithelium health;
(i). maintaining or enhancing gum health;
(j). reducing gum sensitivity;
(k). treating, managing or preventing gingivitis;
(l). treating, managing or preventing periodontitis (gum disease;
(m). treating, managing or preventing inflammation in the oral cavity, for example, gum inflammation;
(n). treating, managing or preventing receding gums; and/or
(o). reducing teeth hypersensitivity.

In a further embodiment, there is provided a non-therapeutic method of treating a condition selected from one or more of the following:
(a). reduction of hypersensitivity of the teeth;
(b). reduction of bad breadth (halitosis);
(c). reduction of plaque accumulation;
(d). reduction of tartar build-up;
(e). treatment, relieving or reduction of dry mouth;
(f). cleaning the teeth and/or oral cavity; and/or
(g). maintaining or enhancing the mouth microbiome; and/or
(h). prevention of stains and/or for whitening teeth;
comprising the administration of a composition of the invention, for example, administration to the oral cavity.

In a further embodiment there is provided use of a composition of the invention for use in subjects with sensitive gums. In a further embodiment, the is provided use of a composition for reducing gum sensitivity.

In a further embodiment, there is provided a composition of the invention for maintaining healthy gums, for the treatment of irritated gums, and for the treatment of patients prone to gingivitis.

In a further embodiment, there is provided a composition of the invention for maintaining or enhancing gum health.

In a further embodiment, there is provided a composition of the invention for reducing or preventing gum bleeding.

In a further embodiment, there is provided a composition of the invention for preventing or reducing gum inflammation.

In a further embodiment, there is provided a composition of the invention for treating irritated gums.

In a further embodiment, there is provided the use of a composition of the invention in a method for enhancing gum longevity.

In a further embodiment, there is provided the use of a composition of the invention in a method for enhancing gum healthspan.

In a further embodiment, there is provided the use of a composition of the invention in a method of facilitating gum regeneration.

In a further embodiment, there is provided the use of a composition of the invention in a method or enhancing or maintaining oral bone health.

In a further embodiment, there is provided the use of a composition of the invention in a method or enhancing or maintaining oral bone health during aging.

In a further embodiment, there is provided the use of a composition of the invention in a method of maintaining or enhancing oral bone density.

In a further embodiment, there is provided the use of a composition of the invention in a method of maintaining or enhancing oral bone density during aging.

In a further embodiment, there is provided the use of a composition of the invention in a method or reducing decreases oral bone health due to aging.

### Aging/Healthspan

According to a further embodiment of the invention, there is provided a composition of the invention for use in the increasing or prolonging healthspan in the oral cavity. Healthspan can be defined as the part of a person's life during which they are generally in good health. Oral health has been linked to increased healthspan and longevity

In a further embodiment of the invention, there is provided a composition of the invention, for slowing aging in the oral cavity.

According to a further embodiment of the invention there is provided a composition of the invention for preventing or reducing oral frailty, associated with aging.

### Medical Treatments:

In one embodiment of the invention, there is provided a composition of the invention for use as a medicament.

In a further embodiment of the invention, there is provided a composition of the invention for use in a disease, disorder or condition characterised by an inadequate mitochondrial activity In a further embodiment of the invention, there is provided a composition of the invention for use in a disease disorder or condition, selected from one or more of the following:
(a). treatment or prevention of erosive tooth demineralization;
(b). reduction or inhibition formation of dental caries;
(c). reduction, repair or inhibition of pre-carious lesions of the enamel, e.g., as detected by quantitative light-induced fluorescence (QLF) or electrical caries measurement (ECM);
(d). reduction or inhibition of demineralization and promote remineralization of the teeth;
(e). inhibit microbial biofilm formation in the oral cavity;
(f). reduction or inhibition of gingivitis;
(g). reduction of gingival inflammation;
(h). reduction of inflammation in the oral cavity;
(i). treatment or prevention of dental implant infections;
(j). promotion of healing of sores or cuts in the mouth;
(k). treatment or prevention of xerostomia (dry mouth);
(l). reduction of levels of acid producing bacteria, in the oral cavity;
(m). increase relative levels of non-cariogenic and/or non-plaque forming bacteria;
(n). increasing of relative levels of arginolytic bacteria;
(o). inhibition of microbial biofilm formation in the oral cavity,
(p). raising and/or maintenance of plaque pH at levels of at least pH 5.5 following sugar (e.g. fructose, glucose, lactose and/or sucrose) challenge;
(q). reducing tooth erosion;
(r). immunization of teeth against cariogenic bacteria; and/or
(s). promotion of systemic health, including cardiovascular health, e.g., by reducing potential for systemic infection via the oral tissues.

In a further embodiment of the invention, there is provided a composition of the invention for use in a disease disorder or condition, selected from one or more of the following:
(a). treatment or prevention of erosive tooth demineralization;
(b). reduction or inhibition of formation of dental caries;
(c). reduction or inhibition of gingivitis;
(d). reduction of inflammation in the oral cavity;
(e). treatment or prevention of dental implant infections;
(f). promotion of healing of sores or cuts in the mouth; and/or
(g). treatment or prevention of xerostomia (dry mouth).

In a further embodiment, there is provided a composition of the invention for use in the treatment of mouth, gum and/or palate inflammation.

### Urolithin Administration/Dosage Regimes

The compositions of the present invention comprise between about 0.1% and about 5% (w/w) of a compound of Formula (I), for example, urolithin A, for example about 0.5% to about 5% (w/w), about 0.5% to about 3% (w/w), about 0.5% to about 2% (w/w), about 1% to about 5% (w/w), about 1% to about 4% (w/w), about 1% to about 3% (w/w), or about 1% to about 2% (w), For example, about 0.5% (w/w), about 1% (w/w), about 1.5% (w/w), about 2% (w/w), about 2.5% (w/w), about 3% (w/w), about 3.5% (w/w), about 4% (w/w), about 4.5% (w/w) or about 5% (w/w). In a further embodiment of the invention, compositions of the invention comprise about 0.1% (w/w), about 0.5% (w/w), about 1% (w/w), about 2% (w/w), about 3% (w/w), about 4% (w/w) or about 5% (w/w)

The methods of the present disclosure would usually require daily administration of the compound of formula (I) or salt thereof, or of a formulation containing the compound or salt, for a period over several months. In some embodiments, the methods may involve administration of the compound of formula (I), or salt thereof, over for example daily for at least 2 weeks, 3 weeks, 4 weeks, 5 weeks, 6 weeks, 8 weeks, 12 weeks, 4 months, 6 months, or for at least a year. In some embodiments, the method comprises administering the compound or salt thereof daily for a period of up to 3 months, up to 6 months, up to 1 year, up to 2 years or up to 5 years. In some embodiments, the method comprises administering the compound or salt daily for a period in the range of from 21 days to 5 years, from 21 days to 2 years, from 21 days to 1 year, from 21 days to 6 months, from 21 days to 12 weeks, from 28 days to 5 years, from 28 days to 2 years, from 28 days to 1 year, from 28 days to 6 months, from 28 days to 4 months, from 28 days to 12 weeks, 6 weeks to 2 years, from 6 weeks to 1 year, from 8 weeks to 1 year, or from 8 weeks to 6 months.

In some embodiments, formulations of the invention comprises a compound of formula (I) or salt thereof (e.g. urolithin A), with a preferred particle size distribution. A particular particle size distribution enables the compound of formula (I) to disperse or dissolve more rapidly. A particular particle size distribution can be achieved by methods established in the art, for example compressive force milling, hamermilling, universal or pin milling, or jet milling (for example spiral jet milling or fluidised-bed jet milling) may be used. Jet milling is especially suitable. Furthermore, a particular particle size distribution may also be directly derived by the use of a particular chemical process. If a particular particle size distribution distribution is used, then preferably the compound has a D₅₀ size of under 100 μm—that is to say that 50% of the compound by mass has a particle diameter size of under 100 μm. More preferably, the compound has a D₅₀ size of under 75 μm, for example under 50 μm, for example under 25 μm, for example under 20 μm, for example under 10 μm. More preferably, the compound has a D₅₀ in the range 0.5-50 μm, for example 0.5 to 20 μm, for example 0.5 to 10 μm, for example 1.0 to 10 μm, for example 1.5 to 7.5 μm, for example 2.8 to 5.5 μm. Preferably, the compound has a D₉₀ size of under 100 μm. More preferably, the compound has a D₅₀ size of under 75 μm, for example under 50 μm, for example under 25 μm, for example under 20 μm, for example under 15 μm. The compound preferably has a Do in the range 5 to 100 μm, for example 5 to 50 μm, for example 5 to 20 μm, for example 7.5 to 15 μm, for example 8.2 to 16.0 μm. Preferably, the compound has a D₁₀ in the range 0.5-1.0 μm. Preferably, the compound of formula (I) or salt thereof (e.g. urolithin A) has a D₉₀ in the range 8.2 to 16.0 μm, a D₅₀ in the range 2.8 to 5.5 μm and a D₁₀ in the range 0.5 to 1.0 μm. In a further embodiment, formulations of the invention comprise compounds of formula (I) without a defined particle size distribution.

In a further embodiment, the compound of formula (I) or salt thereof has a particle size distribution selected from one of the following:
(i) D₅₀ size in the range 0.5 to 50 μm and a D₉₀ size in the range 5 to 100 μm,
(ii) the compound has a D₅₀ size in the range 8.2 to 16.0 μm, a D₅₀ size in the range 2.8 to 5.5 μm and a D₁₀ size in the range 0.5 to 1.0 μm;
(iii) the compound of Formula (I) has a D₅₀ size in the range 0.5 to 20 μm and a D₉₀ size in the range 5 to 50 μm;
(iv) the compound of Formula (I) has a D₅₀ size under 50 μm and a D₉₀ size under 75 μm;
(v) the compound of Formula (I) has a D₅₀ size under 25 μm and a D₉₀ size under 50 μm;
(iv) the compound of Formula (I) has a D₅₀ size under 10 μm and a D₉₀ size under 20 μm;
(v) the compound of Formula (I) has a D₅₀ size under 10 μm and a D₉₀ size under 15 μm; or
(vi) the compound of Formula (I) has a D₅₀ size of 10 μm and a D₉₀ size of 20 μm.

The term “carrier” refers to a diluent, adjuvant, excipient, or vehicle with which the therapeutic or non-therapeutic is administered.

The term ‘essentially free’ refers to compositions having no more than 0.01% by weight of these compounds.

The term ‘excipient’ refers to a substance formulated alongside the active ingredient of a medication or other composition, included, for example, for the purpose of long-term stabilization, bulking up solid formulations that contain potent active ingredients in small amounts (thus often referred to as “bulking agents”, “fillers”, or “diluents”), or to confer a therapeutic enhancement on the active ingredient in the final dosage form, such as facilitating drug absorption, reducing viscosity or enhancing solubility.

The term ‘fluoride source’ refers to any source of fluoride ions, for example, a fluoride salt, which can deliver fluoride to the oral cavity.

The term ‘healthspan’ refers to the number of years that someone lives or can expect to live in reasonably good health. Healthspan may be increased by about 1 to about 20 years, for example, about 1 to about 15 years, for example, 1 to about 10 years, for example 1 to about 8 years, for example, 1 to about 5 years. For example, about 1 year, about 2 years, about 3 years, about 4 years, about 5 years, about 8 years, about 10 years about 15 years or about 20 years.

The term ‘higher alkyl’ refers to an alkyl chain of between about 6 and 30 carbons.

The term ‘oral cavity’ refers to the orifice that air, food, and liquid pass through to keep the human body functioning properly. The oral cavity contains, for example, cheeks, the tongue, teeth, gums salivary glands, palate, alveolar ridge, tonsils, and lips. In one embodiment, the oral cavity excludes the salivary glands.

The term ‘oral care composition’ refers to a composition which has a therapeutic or cosmetic effect in the oral cavity.

The term ‘oral care composition’ refers to a composition, which in the ordinary course of usage, is not intentionally swallowed for purposes of systemic administration of particular cosmetic or therapeutic agents, but is rather retained in the oral cavity for a time sufficient to contact dental surfaces or oral tissues. Examples of oral care compositions include toothpaste, powder, gel, subgingival gel, mousse, foam, breath freshening compositions, chewing gum, lozenges, or denture care or adhesive product, for example, toothpaste, powder, gel, subgingival gel, mousse, foam, breath freshening compositions, chewing gum, or lozenges. ‘Oral care compositions’ do not include products for treating or applying to lips.

The term ‘oral care product’ refers to a device for use in the oral cavity to provide a cosmetic, non-therapeutic or therapeutic effect, Examples of ‘oral care products’ include dental floss, dental tapes, and dental picks (tooth picks). ‘Oral care products’ also include wherein products wherein an oral care composition is incorporated onto strips or films for direct application or attachment to oral surfaces. ‘Oral care products’ do not include products for treating or applying to lips. Incorporation may be applying a layer to the product or impregnating into the matrix of the product.

The term ‘peroxide source’ refers to any orally acceptable peroxide compound (i.e. an oxidizing compound that comprises a bivalent oxygen-oxygen group).

The term “pharmaceutically acceptable” means approved by a regulatory agency of the Federal or a state government or listed in the U.S. Pharmacopeia or other generally recognized pharmacopeia for use in animals, and more particularly in humans.

The term ‘Phosphate’ refers to orally acceptable mono- and polyphosphates, for example, P₁₋₆phosphates, for example monomeric phosphates such as monobasic, dibasic or tribasic phosphate; and dimeric phosphates such as pyrophosphates; and multimeric phosphates, such as tripolyphosphates, tetraphosphates, hexaphosphates and hexametaphosphates (e.g., sodium hexametaphosphate).

The term ‘PVP’ refers to a polymer containing vinylpyrrolidone (also referred to as N-vinylpyrrolidone, N-vinyl-2-pyrrolidione and N-vinyl-2-pyrrolidinone) as a monomeric unit.

The monomeric unit consists of a polar imide group, four non-polar methylene groups and a non-polarmethane group.

The term ‘toothpaste’ includes pastes and gels.

The terms ‘wt %’, ‘weight %’, ‘(w/w)’ and ‘by weight’ have the same meaning expressing the concentration of a substance in a solution or mixture based on its mass relative to the total mass of the solution/mixture.

### EXAMPLES

The invention will now be illustrated with respect to the following non-limiting examples.

Silver nanoparticles (AgNPs) and fluoride (F) are agents widely used in oral medicine and dental practice due to their anti-microbial/anti-cavity properties.

However, exposure of human gingival fibroblasts to AgNPs and F results in increased cellular damage and mitochondrial dysfunction in different cell types derived from the oral cavity. This raises the potential of developing interventions to prevent potentially toxicological effects of AgNPs and F on oral health (Stepniak et al (2014) Int J Nanomedicine 9, 1677-1687; PMC3979695).

For example, after treatment on human gingival cells. AgNPs were mainly found in the mitochondria, leading to increased ROS production, oxidative stress, reduced cell viability, and mitochondrial dysfunctions (Stepniak et al (2014) ibid; PMC3979695). The same detrimental effects were observed after treatment with F. Co-exposure with both AgNPs and F led to even more pronounced cellular health dysfunctions (Stepniak et al (2014) ibid; PMC3979695).

Another study confirmed that NaF induces apoptosis in human gingival fibroblasts (HGF) through both the mitochondria-mediated pathways regulated by the Bcl-2 family and death receptor-mediated pathway (Lee et al (2008) Toxicology 243(3), 340-347; https://doi.org/10.1016/j.tox.2007.10.026).

The negative effects of AgNPs on mitochondrial function were observed also in human fibroblasts (AshaRani et al (2009) ACS Nano 3(2), 279-90; PMID: 19236062).

Fluoride is used to promote enamel mineralization (PMID: 32071168). However, fluoride treatment of enamel cells was shown to negatively affect mitochondrial respiration, elicit mitochondrial membrane depolarization, and disrupt mitochondrial morphology (Aulestia et al (2020) Science Signalling 13, 619; PMID: 32071168).

Chronic fluoride exposure has harmful effects on human health and oral cavity cells (Alvila-Rajas et al (2022) Environmental Toxicology and Pharmacology 94, 103916; https://doi.org/10.1016/j.etap.2022.103916).

Hydrogen peroxide (H₂O₂) is another active commonly used in oral care practises for teeth whitening. However, H₂O₂ is a well-established inducer of reactive oxygen species (ROS) and has been shown to induce a variety of cellular dysfunctions when exposed to gingival cells. For instance, H₂O₂ exposure to gingival cells significantly induced ROS already after 1 h and with increased detrimental effects with exposure up to 24 h (Zgorzynska et al (2015) Arch Oral Biol 60(1) 144-53; PMID: 25455128). Chronic exposure to H₂O₂ in the same cell line was also associated with increased cellular senescence and production of pro-inflammatory cytokines and reduction of collagen production (Furukawa et al (2022) Clin Exp Dent Res 8(4) 939-949; PMC9382052).

### Example 1. Urolithin A Protects Against Cell Death Induced by Silver Nanoparticles (AGNPs) and Fluoride (F) in Human Gingival Fibroblasts (HGF)

### Experimental Outline

The protective effects of urolithin A against cellular dysfunction induced by fluoride (F) and silver nanoparticles (AgNPs) in human gingival fibroblasts (HGF cells) are investigated. In a first experiment, different doses of F, AgNPs or a combination of the two are applied for 24 h to HGFcells. to induce cell death, as described in the method section below and in Stepniak et al (2014) Int J Nanomedicine 9, 1677-1687 (PMC3979695). The doses inducing cellular death are selected for a second experiment. Here, cells are pre-treated with urolithin A at 3 different doses (0.5 uM, 2.5 uM, 25 uM) or DMSO as control for 24 h. Then cells are exposed to the previously selected doses of F, AgNPs or a combination of the two. After 24 h cell viability are measured to assess the ability of UA to rescue cell death induced by F and/or AgNPs.

In a separate experiments exposure of UA of DMSO (as control) are concomitant to the exposure to F, AgNPs and the combination of the two. After 24 h cell viability is measured to assess the ability of UA to rescue cell death induced by F and/or AgNPs.

In another experiment, HGF cells are exposed to selected doses of F and/or AgNPs to assess the impact on mitochondrial respiration. Three different doses of UA (0.5 uM, 2.5 uM, 25 uM) (or DMSO as control) are used to investigate UA's ability to rescue F and AgNPs mediated mitochondrial dysfunction. As above, UA doses or DMSO are applied either 24 h before or at the same time as the F and/or AgNPs exposure. After 24 h mitochondrial respiration is measured by Seahorse respirometer as described in the methods.

### Example 2. Urolithin A Protects Against Cell Death Induced by Silver Nanoparticles (AGNPs) and Fluoride (F) in Enamel Cells

The potential of UA to reverse the detrimental effects of fluoride on mitochondrial in LS8 enamel cells is determined. Cells are pre-treated for 24 h with 3 different doses of UA. Then, cells are treated with NaF for 4 hours, a time period that for which is was shown that fluoride pretreatment results in decreased basal and maximal respiration (Aulestia et al (2020) Science Signalling 13, 619; PMID: 32071168). Mitochondrial respiration is performed to show the beneficial effects of UA against fluoride induced mitochondrial dysfunction.

### Example 3: Randomised Control Trial to Demonstrate Impact of Urolithin a Containing Toothpaste on Oral/Gum Health

The study is a double-blind, placebo-controlled, randomized, parallel group, 3-month home use study in healthy middle-aged volunteers (n=50/arm). Non-smokers with a mean modified gingival index (MGI) score of between 1-3 and at least 20 natural teeth, a minimum of 5 teeth in each quadrant are enrolled in the study. At screening, participants undergo a dental exam by a certified dentist and are issued with a standard fluoride toothpaste (placebo) or standard fluoride toothpaste containing 1% UA along with a toothbrush to use for 13-weeks. Study visits are conducted at baseline, 6-weeks into the study and at the end of the 13-week study period. Gingival health and plaque are re-scored at the 6 and 13-week visits and are compared to baseline scores.

### Example 4. Urolithin A Protects Against Reduced Cell Proliferation Induced by Fluoride (F) in Human Gingival Fibroblasts (HGF)

### Experimental Outline

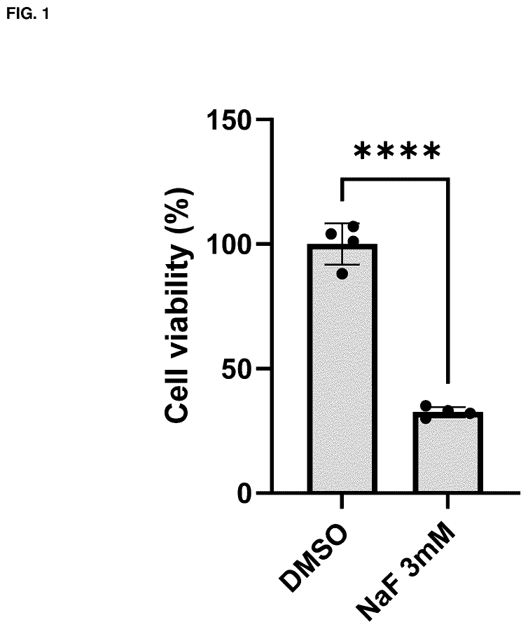

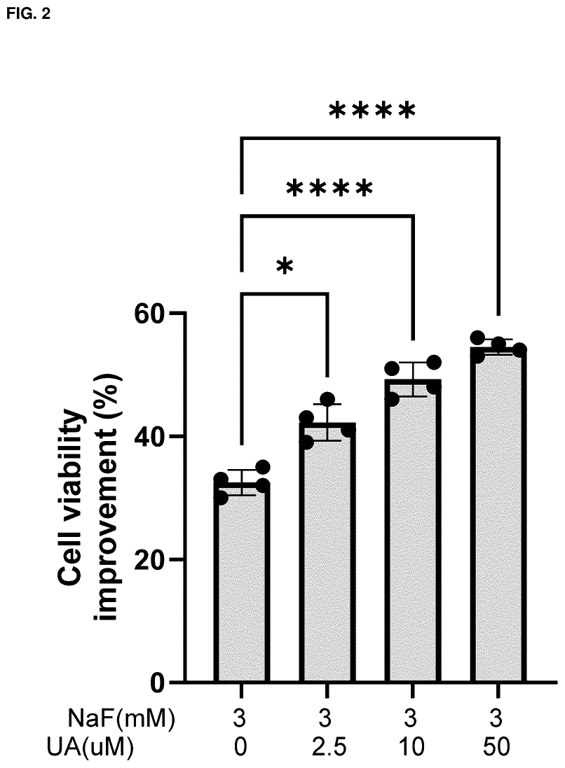

We investigated the protective effects of urolithin A against cellular proliferation inhibition caused by sodium fluoride (NaF) in human gingival fibroblasts (HGF cells). We observed that a dose of 3 mM NaF that was able to significantly reduce cell viability of HGF by 67.5% (FIG. 1). To test urolithin A protective effects in this setting, cells were first pre-treated with urolithin A at 3 different doses (2.5 uM, 10 uM, 50 uM) or DMSO as control for 24 h. Then cells were exposed to the 3 mM NaF with Urolithin A treatments as above. After 24 hours, cell viability was measured to assess the ability of UA to rescue cell proliferation reduction induced by NaF. Results show that the treatment with UA dose dependently rescued F-mediated detrimental cell viability reduction (FIG. 2), with a degree of rescue spanning from +23% cell viability with UA 2.5 uM to +40% cell viability with UA 50 uM. (Table 1).

These data indicate that UA improved gingival cell viability, that is associated with improved gingival tissue regeneration and wound healing.

TABLE 1
Effects of UA on cell viability decline induced by NaF
3 mM, expressed as % change in HGF, related to FIG. 2.

| | % change in | Adjusted P |
|---|---|---|
| Dunnett's multiple comparisons test | viability | Value |
| NaF 3 mM VS NaF 3 mM + UA 2.5 uM | +23% | <0.05 |
| NaF 3 mM vs. Na F 3 mM + UA 10 uM | +34% | <0.001 |
| NaF 3 mM vs. NaF 3 mM + UA 50 uM | +40% | <0.0001 |

### Example 5. Urolithin A Protects Against Inflammation Induced by Sodium Fluoride (NaF) in Human Gingival Fibroblasts (HGF)

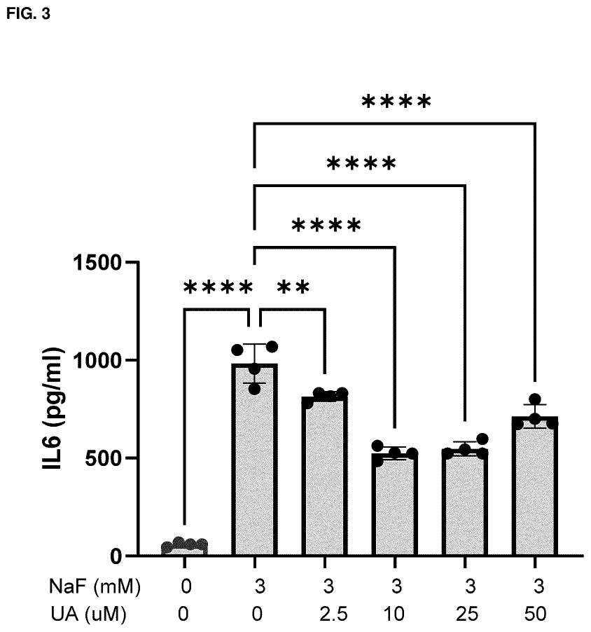

We investigated the protective effects of urolithin A against inflammation induced by sodium fluoride (NaF) in human gingival fibroblasts (HGF cells). A dose of 3 mM F was applied for 24 h to HGF cells to induce inflammation. Inflammation was measured by quantifying the levels of the pro-inflammatory cytokine interleukin 6 (IL-6) in the cell culture supernatant. For the experiment, cells were first pre-treated with urolithin A at 4 different doses (2.5 uM, 10 uM, 25 uM, 50 uM) or DMSO as control for 24 h. Then cells were cotreated with the previously selected dose of NaF and urolithin A. After 24 h, IL-6 was measured in the cell supernatant to assess the ability of Urolithin A to reduce F-induced inflammation. Results indicate that Urolithin A exhibits a protective effect, at all tested concentrations significantly reduced IL-6 levels following treatment with NaF (FIG. 3).

TABLE 2
Effects of UA on IL-6 secretion induced by 3 mM
sodium fluoride (NaF) treatment, expressed as
% change in HGF calculated from data in FIG. 3.

| | % change in | Adjusted P |
|---|---|---|
| Tukey's multiple comparisons test | IL-6 level | Value |
| DMSO vs. NaF | +1590% | <0.0001 |
| NaF 3 mM vs. NaF 3 mM + UA 2.5 uM | −17% | <0.01 |
| NaF 3 mM vs. NaF 3 mM + UA 10 uM | −47% | <0.0001 |
| NaF 3 mM vs. NaF 3 mM + UA 25 uM | −44% | <0.0001 |
| NaF 3 mM vs. NaF 3 mM + UA 50 uM | −27% | <0.0001 |

### Example 6. Urolithin A Protects Against Inflammation in Human Gingival Fibroblasts (HGF) in a Model of Replicative Aging

### Experimental Outline

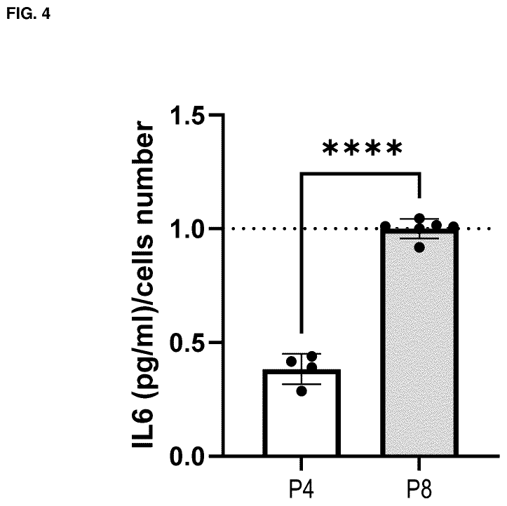

We assessed the ability of Urolithin A to reduce inflammation in a model of replicative gingival cell aging, established based on the publication of Masae Furukawa et al. (2022) Clin Exp Dent Res. 8(4):939-949; (PMC9382052). Here human gingival fibroblasts (HGF) were cultured until passage 8 (P8) to mimic the occurrence of natural aging. Our experiment confirmed results from the published work, showing that levels of the pro-inflammatory cytokine IL-6 were higher in cells at late passages (P8) compared to cells at low passages (P4) (FIG. 4). The increase in IL-6 is in line with the well-established process of inflamm-aging (Franceschi et al., 2017, Trends Endocrinol Metab, 28(3):199-212, PMID: 27789101) that is a low-grade, chronic inflammation accumulating in tissues as we age. This is a key hallmarks of aging (López-Otín et al (2023) Cell 186(2), 243-278; PMID: 36599349) that needs to be countered, to support healthy aging across tissues, including those in the oral cavity.

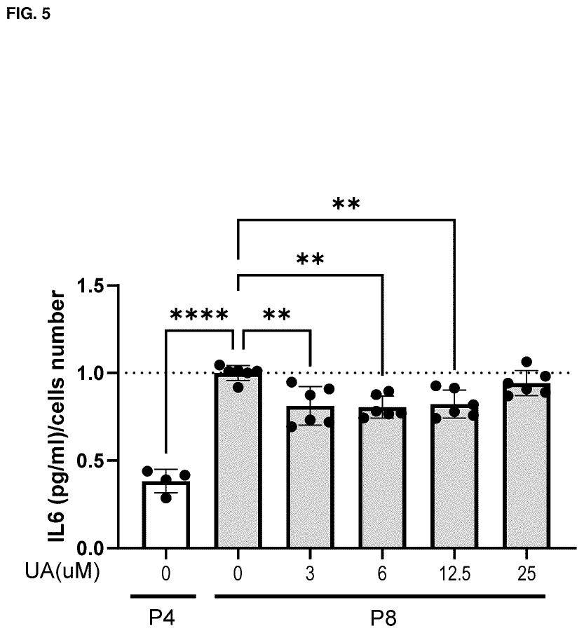

To test the impact of UA on inflamm-aging in the replicative aging model, cells at late passages were treated with UA at 4 doses of 3, 6, 12.5 and 25 uM. After 48 h, IL-6 level was measured in the cell supernatant. Due to difference in cell number between cells at P4 and P8, the IL-6 results were normalized over cell number. The results showed that Urolithin A significantly reduced IL-6 levels (FIG. 5). The combined results indicate that doses between 3 uM to 12.5 uM are optimal to protect gingival cells from both F-mediated inflammation and natural aging mediated inflammation, i.e. inflamm-aging.

TABLE 3
Effects of UA on IL6 secretion induced by replicative
aging, expressed as % change in HGF related to FIG. 5.

| | % change in | Adjusted P |
|---|---|---|
| Tukey's multiple comparisons test | IL-6 level | Value |
| DMSO vs. UA 3 uM | −19% | <0.01 |
| DMSO vs. UA 6 uM | −24% | <0.01 |
| DMSO vs. UA 12.5 uM | −17% | <0.01 |
| DMSO vs. UA 25 uM | −5% | ns |

### Example 7. Urolithin A Protects Against Inflammation Induced by Hydrogen Peroxide (H₂O₂) in Acute Condition in Human Gingival Fibroblasts (HGF)

### Experimental Outline

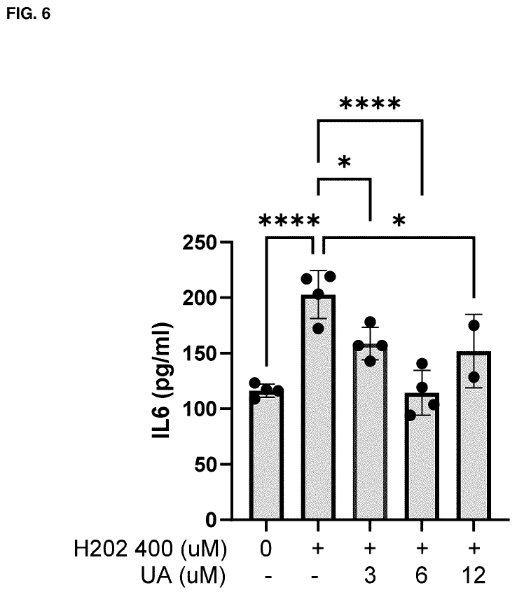

We assessed the ability of Urolithin A to reduce inflammation in gingival fibroblast (HGF) following acute stress induced by H₂O₂. HGF cells were pretreated with Urolithin A at 3 different doses (3 μM, 6 μM, 10 μM) for 24 h. Cells were then cotreated with both Urolithin A at H₂O₂ 400 μM for 24 additional hours. IL-6 was measured in the cell supernatant to assess the ability of Urolithin A to reduce H₂O₂-induced inflammation. Results indicate that Urolithin A exhibits a protective effect, as all tested concentrations significantly reduced IL-6 levels following treatment with H₂O₂(FIG. 6).

TABLE 4
Effects of Urolithin A on IL6 secretion induced
by 400 μM H₂O₂ treatment, expressed as %
change in HGF calculated from data in FIG. 6.

| | % change in | Adjusted |
|---|---|---|
| Tukey's multiple comparisons test | IL-6 level | P Value |
| DMSO vs. H₂O₂ 400 μM | +42% | <0.0001 |
| H₂O₂ 400 μM vs. H₂O₂ 400 μM + UA 3 μM | −21% | <0.05 |
| H₂O₂ 400 μM vs. H₂O₂ 400 μM + UA 6 μM | −44% | <0.0001 |
| H₂O₂ 400 μM vs. H₂O₂ 400 μM + UA 12 μM | −25% | <0.05 |

### Example 8. Urolithin A Protects Against Inflammation Induced by Hydrogen Peroxide (H₂O₂) in Chronic Condition in Human Gingival Fibroblasts (HGF)

### Experimental Outline

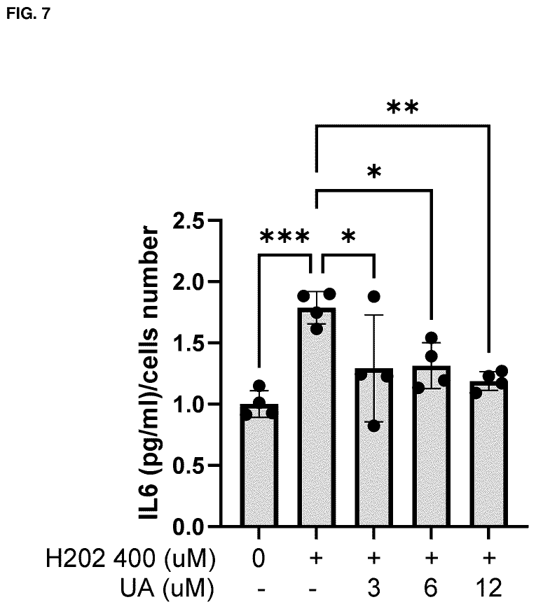

In another experiment, HGF cells were pretreated with Urolithin A at 3 different doses (3, 6, 12 μM) for 24 hours. Cells were then exposed to 400 μM H₂O₂ for 2 hours to induce cellular stress. Following H₂O₂ exposure, cells were incubated for 5 days with either DMSO or Urolithin A at the corresponding doses. IL-6 levels in the culture supernatant were measured by ELISA to evaluate the ability of Urolithin A to rescue inflammation induced by H₂O₂. Due to cell number variability caused by prolonged treatment, IL-6 were normalized over cell number. Results demonstrate that Urolithin A effectively rescues H₂O₂-induced inflammation in a chronic stress model, with all tested concentrations significantly reducing IL-6 secretion (FIG. 7).

TABLE 5
Effects of UA on IL6 secretion induced by H₂O₂ treatment in chronic
condition, expressed as % change in HGF calculated from data
in FIG. 7. HGF were pretreated with Urolithin A for 24 hours,
exposed to 400 μM H₂O₂ for 2 hours and incubate for 5 days
with either DMSO or Urolithin A at the indicated doses.

| | % change in | Adjusted |
|---|---|---|
| Tukey's multiple comparisons test | IL-6 level | P Value |
| DMSO vs. H₂O₂ 400 μM | +44% | <0.001 |
| H₂O₂ 400 μM vs. H₂O₂ 400 mM + UA 3 μM | −28% | <0.05 |
| H₂O₂ 400 μM vs. H₂O₂ 400 mM + UA 6 μM | −26% | <0.05 |
| H₂O₂ 400 μM vs. H₂O₂ 400 mM + UA 12 μM | −33% | <0.01 |

### Example 9. Urolithin A Protects Against Inflammation Induced by Acute Sodium Fluoride (NaF) Exposure in Human Gingival Fibroblasts (HGF)

In another experiment we assessed the protective effect of UA on short-term NaF exposure of gingival cells. This setting is designed to mimic the exposure of cells during the routine application of this molecule in the oral cavity. The typical amount of F contained in oral care products such as toothpastes and mouthwashes ranges approximatively between 500-1000 ppm for children toothpaste, 1000-1500 ppm for adult toothpastes, and 2800-5000 ppm for prescription toothpaste (. This corresponds to an approximate concentration range of sodium fluoride between 10 mM to 120 mM.

First, we investigated the effect of short-term exposure of NaF on inflammation in human gingival fibroblasts (HGF). Inflammation was measured by quantifying the levels of the pro-inflammatory cytokine interleukin 6 (IL-6) in the cell culture supernatant.

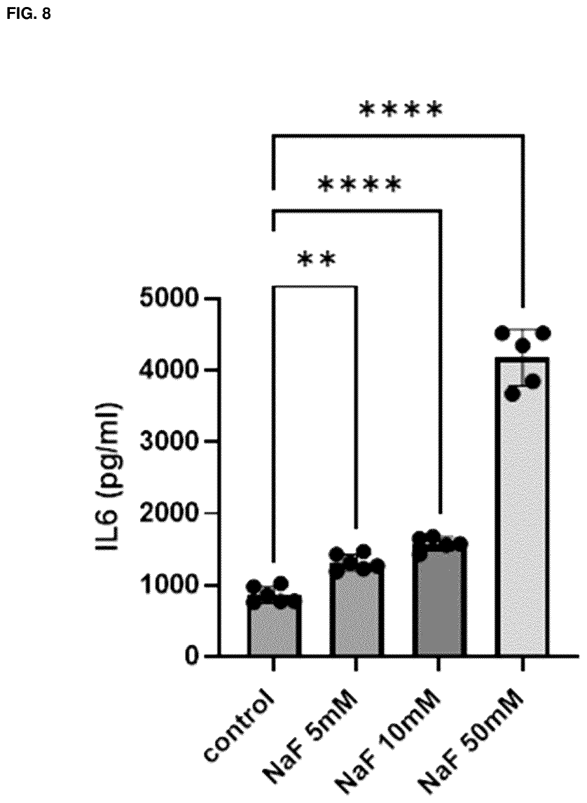

HGF were exposed to NaF 5 mM, 10 mM and 50 mM for 30 minutes. The medium with the treatment was removed and replaced with normal culture medium. Cells were then incubated for further 24 h. Results show that short term exposure of NaF dose-dependently increase the production of the pro-inflammatory cytokine IL-6 (FIG. 8).

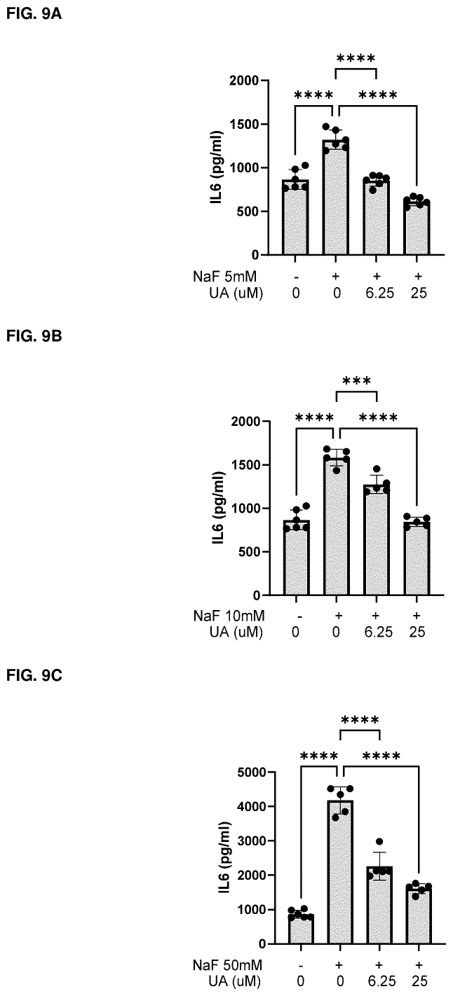

We also investigated the ability of UA to protect against inflammation induced by short-term exposure to NaF. HGF cells were first pre-treated with urolithin A at 6.25 uM and 25 uM or DMSO as control, for 24 h. Then, cells were cotreated with doses NaF at either 5 mM, 10 mM or 50 mM for 30 minutes. The medium with the treatment was removed and replaced with normal culture medium containing again DMSO and UA at the indicated doses. Cells were then incubated for further 24 h. IL-6 was measured in the cell supernatant to assess the ability of Urolithin A to reduce NaF-induced inflammation. Results indicate that Urolithin A exhibits a protective effect at all tested concentrations (FIG. 9). In particular UA at all doses reduces IL-6 levels induces by either NaF 5 mM (FIG. 9A), NaF 10 mM (FIG. 9B) or NaF 50 mM (FIG. 9C). Data show therefore the protective effects of UA on inflammation induced by short term exposure to NaF, the latter used in the same dose ranges commonly present in oral care products.

### Example 10. Urolithin A and Hydroxyapatite have Synergistic Anti-Inflammatory Effects in Human Dermal Fibroblasts (HDF)

Hydroxyapatite (HA) is a naturally occurring mineral form of calcium apatite and the main component of tooth enamel and bone. As concerns about the use of fluoride in toothpaste continue to rise, hydroxyapatite has emerged as a natural, safe, and effective alternative for protecting and repairing tooth enamel.

Beyond its role in oral health, recent evidence suggests that HA may also exert anti-inflammatory effects in fibroblast cells. In particular, HA has been shown in an in vitro study to decrease inflammation in Human Dermal Fibroblast (HDF) (Rakshit et al (2020) Int J Nanomedicine 15, 4943-4956; PMID: 32764927).

In this example, we investigated the potential synergistic effect of Urolithin A (UA) and HA against inflammation induced by a pro-inflammatory cytokine mix (Cytomix, CTX) in HDF. The Cytomix was composed of IL-1b and TNFa at a final concentration of 0.05 ng/mL.

The anti-inflammatory effects of HA, UA and the combination of both compounds were assessed. After seeding, cells were pre-treated for 24 hours with either UA (2.5 uM), HA (500 uM) or the combination of both compounds at the indicated doses. Then, cells were co-exposed to 0.05 ng/ml CTX along with UA, HA or their combination for additional 24 hours. Inflammation was evaluated by quantifying the levels of the pro-inflammatory cytokine interleukin 6 (IL-6) in the cell culture supernatant using IL-6 ELISA. Results were normalised over cell number.

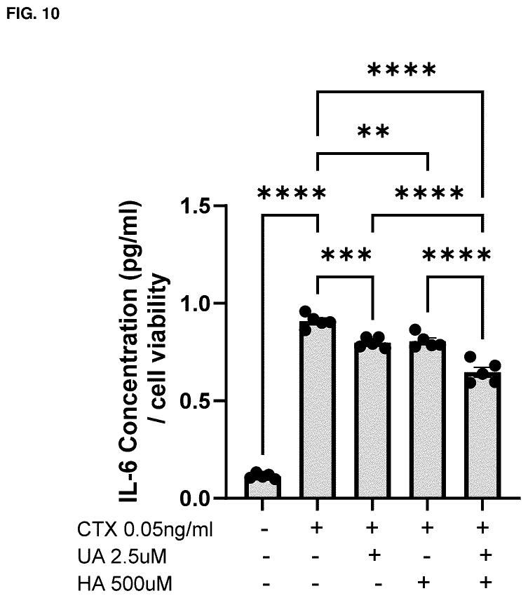

Results indicate that CTX treatment dramatically increases IL-6 levels (FIG. 10). UA and HA treatments reduced CTX-induced IL-6 secretion by 12% and 11% respectively (Table 6). The combination of UA and HA resulted in a statistically significant and synergistic 29% reduction of IL-6 levels compared to CTX. Further confirming the synergy of the two compounds, the combination of UA+HA led to a highly significant decrease of IL-6 by 19% compared to UA treatment alone, and to a highly significant decrease of IL-6 by 20% compared to HA treatment alone.

TABLE 6
Effects of UA, HA and their combination on IL-6 secretion
induced by CTX treatment, expressed as % change
in HDF calculated from data shown in FIG. 10.

| One way ANOVA multiple | % change in | Adjusted |
|---|---|---|
| comparisons test | IL-6 level | P Value |
| DMSO vs. CTX | +702% | <0.0001 |
| CTX vs. UA 2.5 uM | −12% | <0.001 |
| CTX vs. HA 500 uM | −11% | <0.01 |
| CTX vs. HA | −29% | <0.0001 |
| 500 uM + UA 2.5 uM | | |
| CTX + UA 2.5 uM vs. HA | −19% | <0.0001 |
| 500 uM + UA 2.5 uM | | |
| CTX + HA 500 uM vs. HA | −20% | <0.0001 |
| 500 uM + UA 2.5 uM | | |

### Example 11. Urolithin A and Menthol Synergistically Protect Against Basal Inflammation in Human Gingival Fibroblasts (HGF)

Menthol is one of the most commonly used essential oils in oral care products and is also known for its biological activities, including anti-inflammatory and antibacterial effects. Although limited studies have been conducted on gingival cells, Ariane Leite Rozza et al. (2021) Pharmaceutics 13(11):1902; (PMID: 34834317) have investigated the anti-inflammatory and skin wound healing potential of menthol. Based on this publication, and considering the widespread use of menthol in oral care products, we investigated the synergistic effect of Urolithin A and menthol to reduce the basal inflammation in human gingival fibroblast.

After seeding, HGF were exposed to either menthol 1.5 mM, Urolithin A 10 uM or their combination. After 48 hours of treatment, inflammation was evaluated by quantifying the levels of the pro-inflammatory cytokine interleukin 6 (IL-6) in the cell culture supernatant using IL-6 ELISA. Results were normalised over cell number.

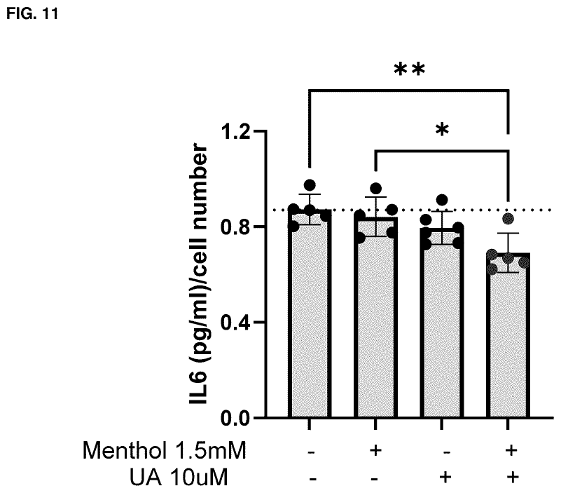

Results indicated that UA and menthol alone decrease IL-6 secretion in these basal, unstressed conditions by 3.5% and 9%, respectively (FIG. 11 and Table 7).

However, the combination of UA and menthol resulted in a statistically significant and synergistic 21% reduction of IL-6 levels compared to DMSO. Further confirming the synergy of the two compounds, the combination of UA+menthol led to a highly significant decrease of IL-6 by 18% compared to menthol treatment alone. These data show how UA can reduce inflammation levels already in unstressed cells and the combination of UA and menthol have synergistic effects.

TABLE 7
Effects of UA, menthol and their combination on IL6 secretion expressed
as % change in HDF calculated from data shown in FIG. 11.

| One way ANOVA multiple | % change in | Adjusted |
|---|---|---|
| comparisons test | IL-6 level | P Value |
| DMSO vs. menthol 1.5 mM | −3.5% | ns |
| DMSO vs UA 10 uM | −9% | ns |
| DMSO vs UA | −21% | <0.01 |
| 10 uM + menthol 1.5 mM | | |
| DMSO + menthol vs UA | −18% | <0.05 |
| 10 uM + menthol 1.5 mM | | |

### Example 12. Urolithin A Protects Against Oxidative Stress Induced by Hydrogen Peroxide (H₂O₂) in Human Gingival Fibroblasts (HGF)

### Experimental Outline

In another experiment, human gingival fibroblast cells are pretreated with Urolithin A at 4 different doses (2.5, 10, 25, 50 μM) for 24 hours. Cells are then exposed to selected doses of H₂O₂ (100, 200, 400 mM) H₂O₂ for 2 to 24 hours to induce oxidative stress. Oxidative stress is measured by MitoSox™ Green (M36008, Invitrogen), following manufacture's instruction. MitoSOX Green Red superoxide indicators is a fluorogenic dyes specifically targeted to mitochondria in live cells. Oxidation of the MitoSOX reagent by mitochondrial superoxide produces bright green fluorescence.

### Example 13. Urolithin A Protects Against Mitochondrial Functions Decline Induced by Fluoride (F) in Human Gingival Fibroblasts (HGF)

### Experimental Outline

In another experiment, HGF cells are exposed to selected doses of F and/or H₂O₂ in the conditions indicated in Example 12 to assess the impact on mitochondrial respiration. Doses of Urolithin A between 2.5 μM to 25 μM (or DMSO as control) are used to investigate Urolithin A's ability to rescue F and H₂O₂ mediated mitochondrial dysfunction. Mitochondrial respiration is measured by Seahorse respirometer as described in the methods.

In a further experiment, HGF cells are exposed to selected doses of F and/or H₂O₂ in the conditions indicated above to assess the impact on the expression of genes associated with mitochondrial function, mitophagy and cellular stress. Doses of Urolithin A between 2.5 μM to 25 μM (or DMSO as control) are used to investigate Urolithin A's ability to rescue F and H₂O₂ mediated alterations in genes associated to mitochondrial and cellular dysfunctions, measured by q-RT-PCR as described in the methods.

### Example 14. Urolithin A and Hydroxyapatite have Synergistic Anti-Inflammatory Effects in Human Dermal Fibroblasts (HDF)

Hydroxyapatite (HA) is a naturally occurring mineral form of calcium apatite and the main component of tooth enamel and bone. As concerns about the use of fluoride in toothpaste continue to rise, hydroxyapatite has emerged as a natural, safe, and effective alternative for protecting and repairing tooth enamel.

Beyond its role in oral health, recent evidence suggests that HA may also exert anti-inflammatory effects in fibroblast cells. In particular, HA has been shown in an in vitro study to decrease inflammation in Human Dermal Fibroblast (HDF) (Rakshit et al (2020) Int J Nanomedicine 15:4943-4956.PMID: 32764927).

In this example, we investigated the potential synergistic effect of Urolithin A (UA) and HA against inflammation induced by a pro-inflammatory cytokine mix (Cytomix, CTX) in HDF. The Cytomix was composed of IL-1b and TNFa at a final concentration of 0.05 ng/mL.

The anti-inflammatory effects of HA, UA and the combination of both compounds were assessed. After seeding, cells were pre-treated for 24 hours with either UA (2.5 uM), HA (500 uM) or the combination of both compounds at the indicated doses. Then, cells were co-exposed to 0.05 ng/ml CTX along with UA, HA or their combination for additional 24 hours. Inflammation was evaluated by quantifying the levels of the pro-inflammatory cytokine interleukin 6 (IL-6) in the cell culture supernatant using IL-6 ELISA. Results were normalised over cell number.

Results indicate that CTX treatment drastically increases IL-6 levels (FIG. 10). UA and HA treatments reduced CTX-induced IL-6 secretion by 12% and 11% respectively (Table 6). The combination of UA and HA resulted in a statistically significant and synergistic 29% reduction of IL-6 levels compared to CTX.

TABLE 8
Effects of UA, HA and their combination on IL6 secretion
induced by CTX treatment, expressed as % change in
HDF calculated from data shown in FIG. 10.

| | % change in | Adjusted |
|---|---|---|
| One way anova multiple comparisons test | IL-6 level | P Value |
| DMSO vs. CTX | +702% | <0.0001 |
| CTX vs. UA 2.5 uM | −12% | <0.001 |
| CTX vs. HA 500 uM | −11% | <0.001 |
| CTX vs. HA 500 uM + UA 2.5 uM | −29% | <0.0001 |

### Methods

### Cell Culture and Treatments

Human gingival fibroblast cells (CRL-2014 cell line) were obtained from the American Type Culture Collection (ATCC-HBT-55) and were maintained as a monolayer culture in T-75 cm² tissue culture flasks. The cells were grown in Dulbecco's Modified Eagle's Medium (ATCC 30-2002), a high glucose medium (4.5 g/L) containing sodium pyruvate (110 mg/L), and were supplemented with 10% foetal bovine serum, 6 μg/mL penicillin-G, and 10 μg/mL streptomycin. Cells were maintained at 37° C. in a humidified atmosphere of 95% O₂, 5% CO₂. When confluent, cells were detached enzymatically with trypsin-EDTA and sub-cultured into a new cell culture flask. The medium was replaced every 2 days.

Normal Human Dermal Fibroblasts (NHDF; C-12302, PromoCell) were cultured as monolayers in T-75 cm² tissue culture flasks. Cells were maintained in Dulbecco's Modified Eagle's Medium (DMEM; Gibco, 21885108), a low glucose formulation (1 g/L) supplemented with sodium pyruvate (110 mg/L), 10% fetal bovine serum (PanBiotech, P30-3033), and 1% penicillin-streptomycin (Biowest, L0022-100). Cultures were incubated at 37° C. in a humidified atmosphere containing 5% CO₂. Upon reaching confluence, cells were detached using trypsin-EDTA and subcultured into new flasks. The culture medium was refreshed every two days.

The sodium fluoride solution was prepared using sodium fluoride (S7920 Sigma Aldrich, St Louis, MO, USA). Urolithin A (Amazentis SA) and Menthol (63660, Sigma Aldrich, St Louis, MO, USA) were dissolved in DMSO and were used at the indicated doses. Hydroxyapatite (677418, Sigma Aldrich, St Louis, MO, USA) was dissolved in water. Chlorhexidine digluconate solution 20% was purchased from Sigma (C9394, Sigma Aldrich, St Louis, MO, USA)

### Cell Viability is Measured by CellTiter Glo

Human gingival fibroblast cells (HGF) were seeded into a 96-well with 5000 cells/well. The day after, HGF cells were treated with Urolithin A (2.5, 10, 25, 50 μM) or DMSO. After 24 hours, HGF cells were exposed to either fluoride (NaF 3 mM) or were co-exposed to Urolithin A at different doses with NaF 3 mM. This assay evaluates mitochondrial activity (assesses cell growth and cell death) and was performed by adding a premixed optimized dye solution to culture wells. Absorbance is recorded at 570 nm (FLUOstar OPTIMA). Results from the treatment groups were calculated as a percentage of control values (unexposed cells) according to the following equation:

$$\% cytotoxicity = ( experimental absorbance [ abs ] 570 nm of exposed cells / abs 570 nm of unexposed cells ) \times 100.$$

### IL-6 Secretion

To assess secretion of the pro-inflammatory cytokine IL-6 inhuman gingival fibroblasts (HGF) cells were seeded into a 96-well with 5000 cells/well. To assess secretion of the pro-inflammatory cytokine IL-6 in human dermal fibroblasts (HDF) cells were seeded into a 96-well with 2500 cells/well. The day after, cells were pre-treated with Urolithin A at the indicated dose or DMSO. After 24 hours of pretreatment, cells were exposed to either NaF, menthol, Cytomix (CTX: IL-1b, TNFa at final concentration of 0.05 ng/mL) or CHX (chlorohexidine) for the indicated treatment duration, or co-exposed with Urolithin A and NaF, CTX, menthol or CHX for 24 hours at different doses. For short term exposure (5 or 30 minutes), the treatment medium was replaced by either Urolithin A or DMSO for the remaining of the 24-hour incubation period. The medium is collected and an ELISA for Interleukin-6 was performed following the instruction provided by the supplier (Proteintech ref. KE10007). Finally, the absorbance at a wavelength of 450 nm was measured using a microplate plate reader (Fluostar Optima, BMG Labtech).

### Replicative Senescence Induction

Human gingival fibroblasts (HGF) are cultured in Dulbecco's Modified Eagle Medium (ATCC 30-2002), a high-glucose medium (4.5 g/L) containing sodium pyruvate (110 mg/L), supplemented with 10% fetal bovine serum (FBS), 6 μg/mL penicillin G, and 10 μg/mL streptomycin. Cells are maintained under standard culture conditions and are passaged upon reaching confluency. Detachment is performed using trypsin-EDTA, and cells are subsequently sub-cultured into new flasks. Replicative aging is induced by continuous passaging, and cells are used for experiments at passage 8. At this stage, cells are treated for 48 hours with either vehicle control (DMSO) or Urolithin A at final concentrations of 3, 6, 12.5, or 25 μM.

### Mitochondrial Respiration

Oxygen consumption is measured using the XF96 Extracellular Flux Analyzer (Agilent).

Once reaching the appropriate confluence, human gingival fibroblast (HGF cells) are shifted in DMEM-based substrate limited medium, containing 0.5 mM glucose, 1 mM GlutaMAX, 0.5 mM carnitine, and are treated with either DMSO (as control), fluoride (F) or Urolithin A. 45 min before the assay, cells are washed two times with Assay Medium (111 mM NaCl, 4.7 mM KCl, 1.25 mM CaCl₂, 2 mM MgSO₄, 1.2 mM NaH₂PO₄, 2.5 mM glucose, 0.5 mM carnitine, oleic acid 5% and 5 mM HEPES adjusted to pH 7.4 at 37° C. on the day of the assay). Cells are then incubated for 30 min in a non-CO₂ incubator. Oxygen consumption rate is determined at maximal level, after the addition of FCCP.

qRT-PCR

RNA from human gingival fibroblast (HGF) cells is extracted using TRIzol (Thermo Scientific, 15596026) and then are transcribed to cDNA by the QuantiTect Reverse Transcription Kit (Qiagen, 205313) following the manufacturer's instructions. The q-RT-PCR reactions are performed using the TaqPath ProAmp master mix (Applied biosystems, A30866) and the expression of selected genes listed below is analysed using the Quantstudio 6 flex (Life Technologies) and following TaqMan probes listed. All quantitative polymerase chain reaction (PCR) results are presented relative to the mean of housekeeping genes (ΔΔCt method). mRNA levels are normalized over RPLP2, ActinB and/or 18S for gene expression for cell and tissue samples.

| Gene name | TaqMan ID |
|---|---|
| 01* PINK1 | Hs00260868_m1 |
| 02* PARK2 | Hs01038318_m1 |
| 03* SDHA C2 | Hs00188166_m1 |
| 04* IL1A | Hs00174092_m1 |
| 07* MMP3 | Hs00968305_m1 |
| 19* ULK1 | Hs00177504_m1 |
| 20* NDUFA1 | Hs00244980_m1 |
| 21* UQCRB | Hs00559884_m1 |
| 22* ESRRA | Hs01067166_g1 |
| 23* RPLP2 | Hs01115128_gH |
| 24* HPRT1 | Hs02800695_m1 |
| 25* BNIP3L | Hs00188949_m1 |
| 26* BECN1 | Hs00186838_m1 |
| 27* PPARA | Hs00947536_m1 |
| 28* IL1b | Hs01555410_m1 |
| 29* IL6 | Hs00174131_m1 |
| 30* MAP1LC3B | Hs00797944_s1 |
| 32* SQSTM1 | Hs00177654_m1 |
| 33* PPIB | Hs00168719_m1 |
| 35* PPARGC1 | Hs01016719_m1 |
| 36* ATP5G1 | Hs04965355_g1 |
| 37* GAPDH | Hs99999905_m1 |
| 38* Actin (ACTB) | Hs99999903_m1 |

### Example 15. Compositions of Toothpastes

TABLE 15a
Composition of Urolithin A toothpaste containing
Fluoride with benefits for oral health

| | | | Health |
|---|---|---|---|
| | | | Benefit |
| | | | groups |
| Component | % or ppm range | Health benefit | (see below) |
| Urolithin A | Range 1: 0.1-10% | Mitochondrial | i. |
| | Range 2: 0.5-2% | function | |
| | Preferred: 1% | enhancer | |
| PQQ | Range 1: 0.005-1% | Mitochondrial | i. |
| | Range 2: 0.01-0.1% | function | |
| | Preferred: 0.05% | enhancer | |
| Fluoride | Range 1: | Restores | ii. |
| | 25-25,000 ppm | enamel/ | |
| | Range 2: | strengthen | |
| | 500-5000 ppm | teeth | |
| | Preferred 1500 ppm | | |
| optionally Ginseng | Range 1: 0.05-5% | Reduces plaque | iii. |
| extract, or one or | Range 2: 0.5-2% | and gingivitis/ | |
| more ginsenosides | Preferred: 1% | reduces | |
| | | inflammation | |
| Optionally | Range 1: 0.05-5% | Reduces plaque | iii. |
| Notoginseng | Range 2: 0.5-2% | and gingivitis/ | |
| extract, or | Preferred: 1% | reduces | |
| one or more | | inflammation | |
| notoginsenosides | | | |
| Zinc ion source | Range 1: 0.05-5% | Tartar control | vi. |
| | Range 2: 0.2-2% | | |
| | Preferred 0.5% | | |
| Hyaluronic acid | Range 1: 0.01-5% | Protects gums | v. |
| | Range 2: 0.1-1% | against | |
| | Preferred 0.2% | bleeding, | |
| | | support tissue | |
| | | regeneration | |
| Quercetin | Range 1: 0.05-5% | Reduces | vi. |
| | Range 2: 0.5-2% | senescence | |
| | Preferred: 1% | | |
| Further inactive | | | |
| carriers | | | |

It is also understood that one or more ingredients of the composition in Table 15a, except urolithin A, can be omitted.

According to a further aspect of the invention there is provided a toothpaste comprising urolithin A in the ranges recited in Table 15a and one of more of the other components, listed in Table 15a, in the ranges recited in Table 15a.

TABLE 15b
15b. Composition of Urolithin A toothpaste containing
Fluoride with benefits for oral health

| | | | Health |
|---|---|---|---|
| | | | Benefit |
| | | | groups |
| Component | % range | Health benefit | (see below) |
| Urolithin A | Range 1: 0.1-10% | Mitochondrial | i. |
| | Range 2: 0.5-2% | function enhancer | |
| | Preferred %: 1% | | |
| Fluoride | Range 1: 25-25,000 ppm | Restores enamel/ | ii. |
| | Range 2: 500-5000 ppm | strengthen teeth | |
| | Preferred 1500 ppm | | |

According to a further aspect of the invention there is provided a toothpaste comprising urolithin A in the ranges recited in Table 15b and fluoride in the ranges recited in Table 15b.

TABLE 15c
15c. Composition of Urolithin A fluoride free
toothpaste with benefits for oral health

| | | | Health |
|---|---|---|---|
| | | | Benefit |
| | | | groups |
| Component | % range | Health benefit | (see below) |
| Urolithin A | Range 1: 0.1-10% | Mitochondrial | i. |
| | Range 2: 0.5-2% | function | |
| | Preferred: 1% | enhancer | |
| PQQ | Range 1: 0.005-1% | Mitochondrial | i. |
| | Range 2: 0.01-0.1% | function | |
| | Preferred: 0.05% | enhancer | |
| Hydroxyapatite | Range 1: 1-50% | Restores | ii. |
| | Range 2: 5-20% | enamel/ | |
| | Preferred: 10% | strengthen | |
| | | teeth | |
| optionally Ginseng | Range 1: 0.05-5% | Reduces plaque | iii. |
| extract, or one or | Range 2: 0.2-2% | and gingivitis/ | |
| more ginsenosides | Preferred: 0.5% | reduces | |
| | | inflammation | |
| optionally | Range 1: 0.05-5% | Reduces plaque | iii. |
| Notoginseng | Range 2: 0.2-2% | and gingivitis/ | |
| extract, or | Preferred 0.5% | reduces | |
| one or more | | inflammation | |
| notoginsenosides | | | |
| Zinc ion source | Range 1: 0.01-5% | Tartar control | vi. |
| | Range 2: 0.1-1% | | |
| | Preferred 0.2% | | |
| Hyaluronic acid | Range 1: 0.01-5% | Protects gums | v. |
| | Range 2: 0.1-1% | against | |
| | Preferred 0.5% | bleeding, | |
| | | support tissue | |
| | | regeneration | |
| Quercetin | Range 1: 0.05-5% | Reduces | vi. |
| | Range 2: 0.5-2% | senescence | |
| | Preferred: 1% | | |
| Further inactive | | | |
| carriers | | | |

It is also understood that one or more ingredients of the composition in Table 12c, except urolithin A, can be omitted.

According to a further aspect of the invention there is provided a fluoride-free toothpaste comprising urolithin A in the ranges recited in Table 15d and one of more of the other components, listed in Table 15d, in the ranges recited in Table 15d.

TABLE 15d
15d. Composition of Urolithin A fluoride free
toothpaste with benefits for oral health

| | | | Health |
|---|---|---|---|
| | | | Benefit |
| | | | groups |
| Component | % range | Health benefit | (see below) |
| Urolithin A | Range 1: 0.1-10% | Mitochondrial | i. |
| | Range 2: 0.5-2% | function enhancer | |
| | Preferred: 1% | | |
| Hydroxyapatite | Range 1: 1-50% | Restores enamel/ | ii. |
| | Range 2: 5-20% | strengthen teeth | |
| | Preferred: 10% | | |

According to a further aspect of the invention there is provided a fluoride-free toothpaste comprising urolithin A in the ranges recited in Table 15d and hydroxyapatite in the ranges recited in Table 15d.

### Example 16. Compositions of Tooth Whitening Toothpaste

TABLE 16a
Composition of Urolithin A tooth whitening toothpaste
containing Fluoride with benefits for oral health

| | | | Health |
|---|---|---|---|
| | | | Benefit |
| | | | groups |
| Component | % or ppm range | Health benefit | (see below) |
| Urolithin A | Range 1: 0.1-10% | Mitochondrial | i. |
| | Range 2: 0.5-2% | function | |
| | Preferred: 1% | enhancer | |
| PQQ | Range 1: 0.005-1% | Mitochondrial | i. |
| | Range 2: 0.01-0.1% | function | |
| | Preferred: 0.05% | enhancer | |
| Fluoride | Range 1: | Restores enamel/ | ii. |
| | 25-25,000 ppm | strengthen teeth | |
| | Range 2: | | |
| | 500-5000 ppm | | |
| | Preferred 1500 ppm | | |
| optionally | Range 1: 0.05-5% | Reduces plaque | iii. |
| Ginseng | Range 2: 0.2-2% | and gingivitis/ | |
| extract, or | Preferred: 0.5% | reduces | |
| one or more | | inflammation | |
| ginsenosides | | | |
| optionally | Range 1: 0.05-5% | Reduces plaque | iii. |
| Notoginseng | Range 2: 0.2-2% | and gingivitis/ | |
| extract, or | Preferred: 0.5% | reduces | |
| one or more | | inflammation | |
| notoginsenosides | | | |
| Zinc ion source | Range 1: 0.01-5% | Tartar control | vi. |
| | Range 2: 0.1-1% | | |
| | Preferred 0.2% | | |
| Hyaluronic acid | Range 1: 0.01-5% | Protects gums | v. |
| | Range 2: 0.1-1% | against bleeding, | |
| | Preferred 0.5% | support tissue | |
| | | regeneration | |
| Quercetin | Range 1: 0.05-5% | Reduces | vi. |
| | Range 2: 0.5-2% | senescence | |
| | Preferred: 1% | | |
| hydrogen | Range 1: 1-40% | Tooth whitening | vii. |
| peroxide | Range 2: 3-10% | | |
| | Preferred: 5% | | |
| Further inactive | | | |
| carriers | | | |

According to a further aspect of the invention there is provided a tooth-whitening toothpaste comprising urolithin A in the ranges recited in Table 16a and one of more of the other components, listed in Table 16a, in the ranges recited in Table 16a.

16b. Composition of Urolithin A tooth whitening toothpaste
without Fluoride with benefits for oral health

| | | | Health |
|---|---|---|---|
| | | | Benefit |
| | | | groups |
| Component | % range | Health benefit | (see below) |
| Urolithin A | Range 1: 0.1-10% | Mitochondrial | i. |
| | Range 2: 0.5-2% | function | |
| | Preferred: 1% | enhancer | |
| PQQ | Range 1: 0.005-1% | Mitochondrial | i. |
| | Range 2: 0.01-0.1% | function | |
| | Preferred: 0.05% | enhancer | |
| Hydroxyapatite | Range 1: 1-50% | Restores enamel/ | ii. |
| | Range 2: 5-20% | strengthen teeth | |
| | Preferred: 10% | | |
| optionally | Range 1: 0.05-5% | Reduces plaque | iii. |
| Ginseng extract, | Range 2: 0.2-2% | and gingivitis/ | |
| or one or more | Preferred: 0.5% | reduces | |
| ginsenosides | | inflammation | |
| optionally | Range 1: 0.05-5% | Reduces plaque | iii. |
| Notoginseng | Range 2: 0.2-2% | and gingivitis/ | |
| extract, or | Preferred: 0.5% | reduces | |
| one or more | | | |
| notoginsenosides | | inflammation | |
| Zinc ion source | Range 1: 0.01-5% | Tartar control | vi. |
| | Range 2: 0.1-1% | | |
| | Preferred 0.2% | | |
| Hyaluronic acid | Range 1: 0.01-5% | Protects gums | v. |
| | Range 2: 0.1-1% | against bleeding, | |
| | Preferred 0.5% | support tissue | |
| | | regeneration | |
| Quercetin | Range 1: 0.05-5% | Reduces | vi. |
| | Range 2: 0.5-2% | senescence | |
| | Preferred: 1% | | |
| hydrogen | Range 1: 1-40% | Tooth whitening | vii. |
| peroxide | Range 2: 3-10% | | |
| | Preferred: 5% | | |
| Further inactive | | | |
| carriers | | | |

It is also understood that one or more ingredients of the composition in Table 16a, except urolithin A, can be omitted.

According to a further aspect of the invention there is provided a tooth-whitening toothpaste comprising urolithin A in the ranges recited in Table 16b and one of more of the other components, listed in Table 16b, in the ranges recited in Table 16b.

16c. Composition of Urolithin A tooth whitening toothpaste

| | | | Health |
|---|---|---|---|
| | | | Benefit |
| | | | groups |
| Component | % range | Health benefit | (see below) |
| Urolithin A | Range 1: 0.1-10% | Mitochondrial | i. |
| | Range 2: 0.5-2% | function enhancer | |
| | Preferred %: 1% | | |
| hydrogen | Range 1: 1-40% | tooth whitening | vii. |
| peroxide | Range 2: 3-10% | | |
| | Preferred: 5% | | |

According to a further aspect of the invention there is provided a tooth-whitening toothpaste comprising urolithin A in the ranges recited in Table 16c and hydrogen peroxide in the ranges recited in Table 16c.

It is understood that one or more ingredients—except urolithin A, of the composition disclosed in Tables of Examples 15 and 16 may be replaced by one or more alternative ingredients selected from the group of ingredients providing the same health benefit as listed below:
i. A mitochondrial function enhancer (for instance, PQQ, Ubiquinone, ubiquinol, Alpha-Lipoic Acid, Nicotinamide Riboside, Dihydronicotinamide riboside, Nicotinamide Mononucleotide, trigonelline, resveratrol)
ii. An agent that restores enamel/strengthen teeth (for instance hydroxyapatite. Further agents include:arginine, xylitol, citric acid, zinc citrate, activated charcoal, calcium sodium phosphosilicate (Novamin), casein phosphopeptide-amorphous calcium phosphate (CPP-ACP), and hydroxyapatite, for example, calcium hydroxyapatite. In one embodiment, the fluoride alternative is hydroxyapatite. In a further embodiment, such ingredients include plant extracts such as extracts from papaya plant, Neem, Azadirachta indica, Tulsi, Ocimum sanctum, Prunus mume, Green and black tea (Camellia sinensi, Chinese Licorice Root, Salvadora persica, Prunusmume, and Hop plant (Humulus lupulus), and Oleic acid, Linoleic acid, epicatechin polymer (Cacao bean husk), proanthocyanidins, phenolic acids, flavonols (Cranberry), and Chlorophyll. In a further embodiments, such ingredient include propolis
iii. An agent to reduce plaque and reduce gingival inflammation, for instance, ginseng extracts, ginsenosides and saponins, aloe vera gel, green tea extract, Epigallocatechin-3-gallate (EGCG), L-Theanine, curcumin, chamomile extract, myrrh extract, cinnamon extract, hydroxytyrosol (for example, from olive leaf extract)
iv. An antibacterial and tartar control agent (for example, Cetylpyridinium Chloride, Delmopinol, essential oils, aloe, xylitol, green tea extract, and/or nisin. Further antibacterial agents include pyridinium compounds (e.g. cetylpyridinium chloride (CPC)), halogenated diphenyl ether (e.g. triclosan), herbal extracts and essential oils (e.g., rosemary extract, tea extract, magnolia extract, thymol, menthol, eucalyptol, geraniol, carvacrol, citral, hinokitol, miswak extract, sea-buckthorn extract), bisguanide antiseptics (e.g., chlorhexidine, alexidine or octenidine), benzalkonium chloride, tetradecylpyridinium chloride (TPC), N-tetradecyl-4-ethylpyridinium chloride (TDEPC)), phenolic antiseptics, hexetidine, octenidine, sanguinarine, povidone iodine, delmopinol, salifluor, metal ions (e.g., zinc salts, for example, zinc chloride, zinc lactate, zinc sulphate, stannous salts, copper salts, iron salts), sanguinarine, propolis and oxygenating agents (e.g., hydrogen peroxide, buffered sodium peroxyborate or peroxycarbonate), phthalic acid and its salts, monoperthalic acid and its salts and esters, ascorbyl stearate, oleoyl sarcosine, alkyl sulphate, dioctyl sulphosuccinate, salicylanilide, domiphen bromide, delmopinol, octapinol and other piperidino derivatives, nicin preparations, chlorite salts; and mixtures of any of the foregoing
v. A protective agent comprising a polymer that forms a barrier layer over gingival tissue to prevent or reduce bleeding (for instance, hyaluronic acid, pectin, arabic gum, carboxymethyl cellulose (CMC), chitosan, xanthan gum, alginate, polyvinylpyrrolidone (PVP), methylcellulose, gelatin, pullulan, and hydroxypropyl methylcellulose (HPMC).
vi. A senolytic agent (for example flavonoids and flavons such apigenin, galangin, rutin, myricetin, fisetin, Berberine, quercetin, dihydroquercetin).
vii. An tooth whitening agent for instance a peroxide source (including for instance hydroperoxides, organic peroxy compounds, peroxy acids, pharmaceutically acceptable salts thereof, and mixtures thereof), Phthalimidoperoxycaproic Acid, baking soda, activated charcoal, papain, bromelain, coconut oil, nano-hydroxyapatite.

### Example 17. Compositions Mouthwash

17a. Composition of Urolithin A mouthwash
with benefits for oral health

| | | | Health |
|---|---|---|---|
| | | | Benefit |
| | | | Groups (see |
| Component | % range | Health benefit | below) |
| Urolithin A | Range 1: 0.1-10% | Mitochondrial | i. |
| | Range 2: 0.5-2% | function | |
| | Preferred: 1% | enhancer | |
| PQQ | Range 1: 0.005-1% | Mitochondrial | i. |
| | Range 2: 0.01-0.1% | function | |
| | Preferred: 0.05% | enhancer | |
| menthol | Range 1: 0.05-2% | Antibacterial | ii. |
| | Range 2: 0.1-1% | agent | |
| | Preferred: 0.4% | | |
| Zinc ion source | Range 1: 0.01-5% | Antibacterial | ii. |
| | Range 2: 0.1-1% | agent | |
| | Preferred 0.2% | | |
| Ginseng extract, | Range 1: 0.1-10% | Reduces plaque | iii. |
| or one or more | Range 2: 0.2-1% | and gingivitis/ | |
| ginsenosides | Preferred 0.5% | reduces | |
| | | inflammation | |
| Notoginseng | Range 1: 0.1-10% | Reduces plaque | iii. |
| extract, or | Range 2: 0.2-1% | and gingivitis/ | |
| one or more | Preferred: 0.5% | reduces | |
| notoginsenosides | | inflammation | |
| Arabic gum | Range 1: 0.01-5% | Protects gums | iv. |
| | Range 2: 0.1-1% | against bleeding, | |
| | Preferred 0.2% | support tissue | |
| | | regeneration | |
| Quercetin | Range 1: 0.05-5% | Reduces | v. |
| | Range 2: 0.5-2% | senescence | |
| | Preferred: 1% | | |
| Further inactive | | | |
| carriers | | | |

According to a further aspect of the invention there is provided a mouthwash comprising urolithin A in the ranges recited in Table 17a and one of more of the other components, listed in Table 17a, in the ranges recited in Table 17a.

17b. Composition of Urolithin A mouthwash
with benefits for oral health

| | | | Health |
|---|---|---|---|
| | | | Benefit |
| | | | Groups |
| Component | % range | Health benefit | (see below) |
| Urolithin A | Range 1: 0.1-10% | Mitochondrial | i. |
| | Range 2: 0.5-2% | function | |
| | Preferred %: 1% | enhancer | |
| menthol | Range 1: 0.05-2% | Antibacterial | ii. |
| | Range 2: 0.1-1% | agent | |
| | Preferred: 0.4% | | |
| Further inactive | | | |
| carriers | | | |

According to a further aspect of the invention there is provided a mouthwash comprising urolithin A in the ranges recited in Table 17b and menthol in the ranges recited in Table 17b.

It is understood that one or more ingredients of the composition disclosed in all Tables 17a and 17b may be replaced by one or more alternative ingredients selected from the group of ingredients providing the same health benefit as listed below:
i. A mitochondrial function enhancer (for instance, PQQ, Ubiquinone, ubiquinol, Alpha-Lipoic Acid, Nicotinamide Riboside, Dihydronicotinamide riboside, Nicotinamide Mononucleotide, trigonelline, resveratrol)
ii. An antibacterial agent (for instance, Cetylpyridinium Chloride, Delmopinol, aloe, xylitol, and/nisin). Further antibacterial agents include pyridinium compounds (e.g. halogenated diphenyl ether (e.g. triclosan), herbal extracts and essential oils (e.g., rosemary extract, tea extract, magnolia extract, thymol, menthol, eucalyptol, geraniol, carvacrol, citral, hinokitol, miswak extract, sea-buckthorn extract), bisguanide antiseptics (e.g., chlorhexidine, alexidine or octenidine), benzalkonium chloride, tetradecylpyridinium chloride (TPC), N-tetradecyl-4-ethylpyridinium chloride (TDEPC)), phenolic antiseptics, hexetidine, octenidine, sanguinarine, povidone iodine, delmopinol, salifluor, metal ions (e.g., zinc salts, for example, zinc chloride, zinc lactate, zinc sulphate, stannous salts, copper salts, iron salts), sanguinarine, propolis and oxygenating agents (e.g., hydrogen peroxide, buffered sodium peroxyborate or peroxycarbonate), phthalic acid and its salts, monoperthalic acid and its salts and esters, ascorbyl stearate, oleoyl sarcosine, alkyl sulphate, dioctyl sulphosuccinate, salicylanilide, domiphen bromide, delmopinol, octapinol and other piperidino derivatives, nicin preparations, chlorite salts; and mixtures of any of the foregoing
iii. An agent to reduce plaque and reduce gingival inflammation, for instance, ginseng extracts, ginsenosides and saponins, aloe, green tea extract, Epigallocatechin-3-gallate (EGCG), L-Theanine, aloe, curcumin, chamomile extract, myrrh extract, cinnamon extract, hydroxytyrosol (for example, from olive leaf extract)
iv. A protective agent comprising a polymer that forms a barrier layer over gingival tissue to prevent or reduce bleeding (for instance, hyaluronic acid, pectin, arabic gum, carboxymethyl cellulose (CMC), chitosan, xanthan gum, alginate, polyvinylpyrrolidone (PVP), methylcellulose, gelatin, pullulan, and hydroxypropyl methylcellulose (HPMC).
v. A senolytic agent (for example flavonoids and flavons such apigenin, galangin, rutin, myricetin, fisetin, Berberine, quercetin, dihydroquercetin);

### STATEMENTS OF INVENTION

1. An oral care composition comprising:
   a). a compound of formula (I) or a salt thereof:

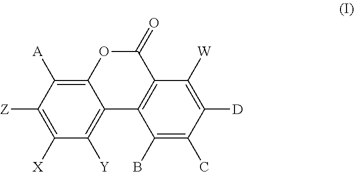

wherein:
A, B, C and D are each independently selected from H and OH;
W, X and Y are each independently selected from H and OH; and
Z is selected from H and OH.
2. An oral care composition, as claimed in statement 1, wherein the composition is selected from a toothpaste, a powder, a mouthwash, an oral spray, an oral gel, a mouth rinse, a chewing gum, or a lozenge, for example, a toothpaste, an oral gel and a mouthwash.
3. An oral care composition, as claimed in statement 2 wherein the composition is a toothpaste or oral gel.
4. An oral care composition, as claimed in statement 2 wherein the composition is a mouthwash.
5. An oral care composition, as claimed in statement 1, wherein the composition is applied to an oral care product, for example, dental floss, dental tape, dental strips or dental sticks.
6. An oral care composition as claimed in any one of statements 1 to 5 wherein the composition further comprises:
   b). a fluoride source.
7. An oral care composition, as claimed in statement 6, wherein the fluoride source is selected from: sodium fluoride, sodium monofluorophosphate, and stannous fluoride, or a mixture one or more thereof.
8. An oral care composition, as claimed in any one of statements 1 to 5, essentially free of a fluoride source.
9. An oral care composition, as claimed in statement 8 further comprising one or more ingredients selected from arginine, xylitol, citric acid, zinc citrate, activated charcoal, calcium sodium phosphosilicate (Novamin), casein phosphopeptide-amorphous calcium phosphate (CPP-ACP), and hydroxyapatite, for example, calcium hydroxyapatite.
10. A oral care composition as claimed in any one of the preceding statements wherein the composition further comprises
   c). an abrasive.
11. An oral care composition as claimed in statement 10 wherein the abrasive is selected from a silica abrasive.
12. An oral care composition as claimed in statement 10 or statement 11 wherein the abrasive comprises a calcium phosphate abrasive, e.g., tricalcium phosphate (Ca₃(PO₄)₂), hydroxyapatite (Ca₁₀(PO₄)₆(OH)₂), or dicalcium phosphate dihydrate (CaHPO₄·2H₂0; DiCal) or calcium pyrophosphate; calcium carbonate abrasive; or abrasives such as sodium metaphosphate, potassium metaphosphate, aluminium silicate, calcined alumina, bentonite or other siliceous materials, or combinations thereof.
13. A composition, as claimed in any one of the preceding statements for use as a medicament.
14. A composition, as claimed in any one of statements 1 to 12 for use in a disease, disorder, or condition characterised by inadequate mitochondrial activity
15. A non-therapeutic method of treating a condition selected from one or more of the following:
   (a) reduction of hypersensitivity of the teeth;
   (b) reduction of bad breadth (halitosis);
   (c) reduction of plaque accumulation;
   (d) reduction of tartar build-up;
   (e) treatment, relieving or reduction of dry mouth;
   (f) cleaning the teeth and/or oral cavity; and/or
   (g) prevention of stains and/or for whitening teeth. comprising the administration of a composition as claimed in any one of claims 1 to 12, for example, administration to the oral cavity.
16. A composition, as claimed in any one of statements 1 to 12 for use in the treatment or prevention of a disorder selected from:
   (a). erosive tooth demineralization;
   (b). dental caries;
   (c). gingivitis;
   (d). inflammation in the oral cavity, for example, gum inflammation;
   (e). dental implant infections;
   (f). sores or cuts in the mouth;
   (g). xerostomia (dry mouth).
17. A composition as claimed in any one of statement 1 to 12 for enhancing oral healthspan.
18. A composition as claimed in any one of statements 1 to 12 for maintaining or enhancing gum health.
19. A composition, as claimed in any one of the preceding statement, wherein the compound of Formula (I) is urolithin A, urolithin B, urolithin C and/or urolithin D, for example, urolithin A.
20. A composition, as claimed in statement 19, wherein the compound of Formula (I) is urolithin A.

### EQUIVALENTS

The invention has been described broadly and generically herein. Those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention. Further, each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

### INCORPORATION BY REFERENCE

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right physically to incorporate into this application any and all materials and information from any such articles, patents, patent applications, or other physical and electronic document.

1. An oral care composition comprising:
a) a compound of formula (I) or a salt thereof: 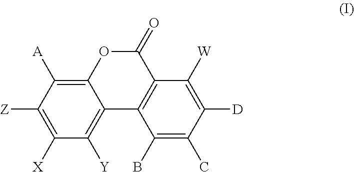
wherein:
A, B, C and D are each independently selected from H and OH;
W, X and Y are each independently selected from H and OH; and
Z is selected from H and OH; and
b) an oral care composition additive, wherein the oral care composition additive is selected from one or more of a fluoride source, a fluoride replacement, an antiseptic and a whitening agent.

2. The oral care composition of claim 1, wherein the oral care composition additive is a fluoride source, for example, wherein the fluoride source is selected from one or more of:
a) One or more fluoride salts, for example, sodium fluoride, stannous fluoride, potassium fluoride, potassium stannous fluoride, indium fluoride, zinc fluoride, ammonium fluoride, stannous chlorofluoride, sodium monofluorophosphate, sodium fluorosilicate, ammonium fluorosilicate, amine fluoride, titanium fluoride, hexafluorosulphate, calcium fluoride, ammonium fluoride and aluminium fluoride; or
b) a fluoride source is selected from hexadecyl ammonium fluoride, 3-(N-hexadecyl-N-2-hydroxy-ethylammonio) ammonium difluoride, N,N N-Tris(polyoxyethylene)-N-hexadecyl-propylenediaminedihydrofluoride disodium hexafluorosilicate, dipotassiumhexafluorosilicate, ammonium hexafluorosilicate, magnesium hexafluorosilicate and ammonium fluorophosphates.

3. The oral care composition of claim 2, wherein the fluoride source is selected from sodium fluoride, sodium monofluorophosphate, and/or stannous fluoride.

4. The oral care composition of claim 1, wherein the oral care composition additive is a fluoride replacement, for example, wherein the fluoride replacement is selected from one or more of arginine, xylitol, citric acid, zinc citrate, activated charcoal, calcium sodium phosphosilicate (Novamin), casein phosphopeptide-amorphous calcium phosphate (CPP-ACP), and hydroxyapatite, for example, calcium hydroxyapatite.

5. The oral care composition of claim 1, wherein the oral care composition additive is an antiseptic, for example. one or more of essential oils (e.g., rosemary extract, tea extract, magnolia extract, thymol, menthol, eucalyptol, geraniol, carvacrol, citral, hinokitol, miswak extract, sea-buckthorn extract), and/or one or more zinc salts.

6. The oral care composition of claim 1, wherein the oral care composition additive is a whitening agent, for example, a peroxide source, such as hydrogen peroxide, sodium peroxide and/or carbamide peroxide.

7. The oral care composition of claim 1, wherein the composition is selected from a toothpaste, a powder, a mouthwash, an oral spray, an oral gel, a mouth rinse, a chewing gum, a tooth whitening pen, or a lozenge, for example, a toothpaste, an oral gel and a mouthwash.

8. The oral care composition of claim 1, wherein the composition is applied to an oral care product, for example, dental floss, dental tape, dental strips or dental sticks.

9. The oral care composition of claim 1, wherein the composition further comprises an abrasive.

10. The oral care composition of claim 9, wherein the abrasive is selected from a silica abrasive.

11. The oral care composition of claim 9, wherein the abrasive is tricalcium phosphate (Ca₃(PO₄)₂), hydroxyapatite (Ca₁₀(PO₄)₆(OH)₂), dicalcium phosphate dihydrate (CaHPO₄·2H₂O), calcium pyrophosphate sodium metaphosphate, potassium metaphosphate, aluminium silicate, calcined alumina, bentonite or other siliceous materials, or combinations thereof.

12. (canceled)

13. (canceled)

14. (canceled)

15. The oral care composition of claim 1, wherein the compound of Formula (I) is urolithin A, urolithin B, urolithin C and/or urolithin D.

16. The oral care composition of claim 15, wherein the compound of Formula (I) is urolithin A.

17. The oral care composition of claim 1, comprising
a) about 0.1% to about 10% (w/w) of a compound of formula (I);
b) about 25 to about 25,000 ppm of a fluoride source; and
c) one or more inactive carriers.

18. The oral care composition of claim 1, comprising
a) about 0.1% to about 10% (w/w) of a compound of formula (I);
b) about 1% to about 50% (w/w) of a fluoride replacement; for example, hydroxyapatite and
c) one or more inactive carriers.

19. The oral care composition of claim 1, comprising
a) about 0.1% to about 10% (w/w) of a compound of formula (I);
b) about 1% to about 40% (w/w) of whitening agent, such as a peroxide source, for example, H₂O₂; and
c) one or more inactive carriers.

20. The oral care composition of claim 1, comprising
a) about 0.1% to about 10% (w/w) of a compound of formula (I);
b) about 0.05% to about 2% menthol; and
c) one or more inactive carriers.

21. The oral care composition of claim 1, further comprising one or more of the following:
i) about 0.005% to about 1% (w/w) PQQ;
ii) about 0.05% (w/w) to about 5% (w/w) ginseng extract, or one or more ginsenosides;
iii) about 0.05% (w/w) to about 5% (w/w) notoginseng extract, or one or more notoginsenosides;
iv) about 0.01% to about 5% (w/w) zinc ion source;
v) about 0.01% to about 5% (w/w) hyaluronic acid;
vi) about 0.01% to about 5% (w/w) pectin; and
vii) about 0.05% to about 5% (w/w) quercetin.

22. A method of treating oral disease, disorder, or condition characterized by inadequate mitochondrial activity, comprising administering to the oral cavity of a subject in need thereof an effective amount of oral care composition of claim 1.

23. A non-therapeutic method for:
(a) enhancing oral and gum healthspan and/or longevity;
(b) enhancing tongue healthspan and/or longevity;
(c) maintaining or enhancing oral muscosa health;
(d) maintaining or enhancing oral epithelium health;
(e) maintaining or enhancing gum health;
(f) reducing gum sensitivity;
(g) treating or preventing gingivitis;
(h) treating or preventing periodontitis (gum disease);
(u) treating or preventing inflammation in the oral cavity;
(j) treating or preventing receding gums; and/or
(k) reducing teeth hypersensitivity;
comprising administering to the oral cavity of a subject in need thereof an effective amount of an oral care composition of claim 1.
